(19) United States
(12) Patent Application Publication
(10) Pub. No.: US 2023/0390565 A1
(43) Pub. Date: Dec. 7, 2023

(54) WMS-WCD HAVING UI WITH VOICE PROMPT TO RENEW BATTERY

(71) Applicant: West Affum Holdings Corp., Grand Cayman (KY)

(72) Inventors: Stacy Taylor, Kingston, WA (US); Cameron G. Pollock, Kirkland, WA (US); Traci S. Umberger, Kirkland, WA (US); Kenneth F. Cowan, Everett, WA (US); Gregory T. Kavounas, Bellevue, WA (US)

(21) Appl. No.: 17/830,998

(22) Filed: Jun. 2, 2022

(51) Int. Cl. A61N 1/378; A61N 1/39; A61N 1/02

(52) U.S. Cl. CPC A61N 1/3787; A61N 1/3975; A61N 1/3993; A61N 1/025; A61N 1/3904

(57) ABSTRACT

A wearable medical systems (“WMS”) implements a wearable cardioverter defibrillator (“WCD”) by including therapy electrodes coupled to a unit with electronic components, all of which are carried or worn by a patient. A charger is also provided, along with at least two rechargeable batteries; one of the batteries can be powering the unit while the other is being recharged by the charger. The unit may output a prompt to the patient to swap the batteries when the battery in the unit reaches a low-charge condition, or when other conditions are met.

### BACKGROUND

A wearable medical system (“WMS”) is an advanced form of a medical system. A WMS typically includes one or more wearable components that a patient can wear or carry, and possibly other components that can be portable, or stationary such as base station and/or an electric charger. The WMS may also include one or more associated software packages, such as software applications (“apps”), which can be hosted by the wearable component, and/or by a mobile device, and/or by a remote computer system that is accessible via a communications network such as the internet, and so on.

A WMS typically includes a sensor that can sense when a parameter of the patient is problematic, and cause the WMS to initiate an appropriate action. The appropriate action could be for the WMS to communicate with the patient or even with a bystander, to transmit an alert to a remotely located clinician, and to even administer treatment or therapy to the patient by itself. A WMS may actually include more than one sensor, which may sense more than one parameter of the patient. The multiple parameters may be used for determining whether or not to administer the treatment or therapy, or be suitable for detecting different problems and/or for administering respectively different treatments or therapies to the patient.

A WMS may also include the appropriate components for implementing a wearable cardioverter defibrillator (“WCD”), a pacer, and so on. Such a WMS can be for patients who have an increased risk of sudden cardiac arrest (“SCA”). In particular, when people suffer from some types of heart arrhythmias, the result may be that blood flow to various parts of the body is reduced. Some arrhythmias may result in SCA, which can lead to death very quickly, unless treated within a short time, such as 10 minutes. Some observers may have thought that SCA is the same as a heart attack, but it is not. For such patients, an external cardiac defibrillator can deliver a shock through the heart, and restore its normal rhythm. The problem is that it is hard for an external cardiac defibrillator to be brought to the patient within that short time. One solution, therefore, is for such patients to be given a WMS that implements a WCD. This solution is at least temporary and, after a while such as two months, the patient may instead receive a surgically implantable cardioverter defibrillator (“ICD”), which would then become a permanent solution.

A WMS that implements a WCD typically includes a harness, vest, belt, or other garment that the patient is to wear. The WMS system further includes additional components that are coupled to the harness, vest, or other garment. Alternately, these additional components may be adhered to the patient's skin by adhesive. These additional components include a unit that has a defibrillator, and sensing and therapy electrodes. When the patient wears this WMS, the sensing electrodes may make good electrical contact with the patient's skin and therefore can help sense the patient's Electrocardiogram (“ECG”). If the unit detects a shockable heart arrhythmia from the ECG, then the unit delivers an appropriate electric shock to the patient's body through the therapy electrodes. The shock can pass through the patient's heart and may restore its normal rhythm, thus saving their life.

All subject matter discussed in this Background section of this document is not necessarily prior art, and may not be presumed to be prior art simply because it is presented in this Background section. Plus, any reference to any prior art in this description is not, and should not be taken as, an acknowledgement or any form of suggestion that such prior art forms parts of the common general knowledge in any art in any country. Along these lines, any recognition of problems in the prior art discussed in this Background section or associated with such subject matter should not be treated as prior art, unless expressly stated to be prior art. Rather, the discussion of any subject matter in this Background section should be treated as part of the approach taken towards the particular problem by the inventors. This approach in and of itself may also be inventive.

### BRIEF SUMMARY

The present description gives instances of wearable medical systems (“WMS”) that implement a wearable cardioverter defibrillator (“WCD”), storage media that may store programs, and methods, the use of which may help overcome problems and limitations of the prior art.

A wearable medical system (“WMS”) implements a wearable cardioverter defibrillator (“WCD”) by including therapy electrodes coupled to a unit with electronic components, all of which are carried or worn by a patient. A charger is also provided, along with at least two rechargeable batteries; one of the batteries can be powering the unit while the other is being recharged by the charger. The unit may output one or more prompts to the patient about the batteries when certain conditions are met.

In first embodiments, the charger sequentially communicates to the unit the charging level of the battery it is charging. If the unit detects a charger-not-charging condition being met, it may output a caution prompt to the patient to ensure that the second battery is being charged by the charger. An advantage or benefit can be that a situation is avoided where, when the battery in the unit becomes low on charge, the other battery has inadvertently not been charged, leaving the patient unprotected.

In second embodiments, a prompt is a low-charge prompt when the battery in the unit reaches a low-charge condition, but the low-charge prompt is not sent if the battery in the charger is charged even less than the battery in the unit. Instead, other measures may be taken.

In third embodiments, a prompt is a low-charge prompt when the battery in the unit reaches a low-charge condition, but the low-charge condition is updated if a capacitor in the unit is charged and/or discharged in the interim. An advantage or benefit can be that the battery is better prepared for situations where a patient emergency requires multiple shocks.

In fourth embodiments, the prompt is output at an opportune moment earlier than the low-charge prompt. An advantage or benefit can be that the patient may swap the batteries at the opportune moment, and therefore not have to swap them at a later time when it is inconvenient or impossible to swap them, such while sleeping at night, or being away from home.

In fifth embodiments, the unit further identifies the third battery as unplugged from the unit and from the charger, determines for how long it has been unplugged, and provides a reminder prompt to recharge it as well when a condition is met. An advantage or benefit can be that the patient may keep additional reserve batteries in places remote from the charger, for example in an office or the trunk of a car, free from the fear that their charge will decay and they will become unprotected.

As such, it will be appreciated that results of embodiments are larger than the sum of their individual parts, and have utility.

These and other features and advantages of the claimed invention will become more readily apparent in view of the embodiments described and illustrated in this specification, namely in this written specification and the associated drawings.

### BRIEF DESCRIPTION OF THE DRAWINGS

FIG. 1 is a diagram of sample components of a wearable medical system (“WMS”) that implements a wearable cardioverter defibrillator (“WCD”), and which is made according to embodiments.

FIG. 2A is a diagram showing a view of the inside of a sample garment embodiment that can be a support structure of a WMS that implements a WCD, such as that of FIG. 1.

FIG. 2B is a diagram showing a view of the outside of the sample garment of FIG. 2A.

FIG. 2C is a diagram showing a front view of how the sample garment of FIGS. 2A and 2B is intended to be worn by a patient.

FIG. 2D is a diagram showing a back view of how the sample garment of FIGS. 2A and 2B is intended to be worn by a patient.

FIG. 3 is a diagram showing a partial front view of another patient wearing a sample garment embodiment of an alternate style as worn by a patient, and which can be a support structure of a WMS that implements a WCD such as that of FIG. 1.

FIG. 4 is a diagram showing sample embodiments of electronic components of a WMS that implements a WCD, and which can be used with the garment of FIG. 2A or of FIG. 3.

FIG. 5 is a diagram showing sample components of a unit of FIG. 1, which is made according to embodiments.

FIG. 6 is a diagram of sample components of a single wearable medical system (“WMS”) that implements a WCD, according to embodiments.

FIG. 7 is a diagram showing sample components of a charger and of a unit of a WMS that implements a WCD according to first embodiments.

FIG. 8 is a diagram showing time evolutions of battery levels of the batteries in the first embodiments of FIG. 7 for sample scenarios.

FIG. 9 is a diagram similar to FIG. 8, and which shows detailed time evolutions of battery levels in a sample scenario.

FIG. 10 is a flowchart for illustrating sample methods according to first embodiments.

FIG. 11 is a flowchart for illustrating sample methods according to embodiments for performing an operation of the methods of FIG. 10.

FIG. 12 is a flowchart for illustrating sample methods according to embodiments for performing another operation of the methods of FIG. 10.

FIG. 13 is a diagram showing sample components of a charger and of a unit of a WMS that implements a WCD according to second embodiments.

FIG. 14 is a diagram showing time evolutions of battery levels of the batteries in the second embodiments of FIG. 13 for a specific scenario.

FIG. 15 is a flowchart for illustrating sample methods according to second embodiments.

FIG. 16 is a diagram showing sample components of a charger and of a unit of a WMS that implements a WCD according to third embodiments.

FIG. 17 is a diagram showing time evolutions of battery levels of the batteries in the third embodiments of FIG. 16 for a sample scenario.

FIG. 18 is a flowchart for illustrating sample methods according to third embodiments.

FIG. 19 is a diagram showing sample components of a charger and of a unit of a WMS that implements a WCD according to fourth embodiments.

FIG. 20 is a diagram showing time evolutions of battery levels of the batteries in the fourth embodiments of FIG. 19 for a sample scenario.

FIG. 21 is a flowchart for illustrating sample methods according to fourth embodiments.

FIG. 22 is a diagram showing sample components of a charger and of a unit of a WMS that implements a WCD according to fifth embodiments.

FIG. 23 is a diagram showing sample components and operations of components of the fifth embodiments of FIG. 22.

FIG. 24 is a diagram showing time evolutions of battery levels of the batteries in the fifth embodiments of FIG. 22 for a sample scenario.

FIG. 25 is a flowchart for illustrating sample methods according to fifth embodiments.

FIG. 26 is a diagram showing how regenerative charging circuitry can be used for a WMS implementing a WCD according to embodiments.

FIG. 27 is a diagram replicated from a prior art reference that can be adapted for implementing the embodiments of FIG. 26.

FIG. 28 is a diagram replicated from a prior art reference that can be adapted for implementing the embodiments of FIG. 26.

FIG. 29 is a diagram replicated from a prior art reference that can be adapted for implementing the embodiments of FIG. 26.

### DETAILED DESCRIPTION

As has been mentioned, the present description is about wearable medical systems (“WMS”) that implement a wearable cardioverter defibrillator (“WCD”), storage media that may store programs, and methods. Embodiments are now described in more detail.

A wearable medical system (“WMS”) that implements a wearable cardioverter defibrillator (“WCD”) according to embodiments may protect an ambulatory patient by electrically restarting their heart if needed. Such a WMS may have a number of components. These components can be provided separately as modules that can be interconnected, or can be combined with other components, and so on. Examples are now described.

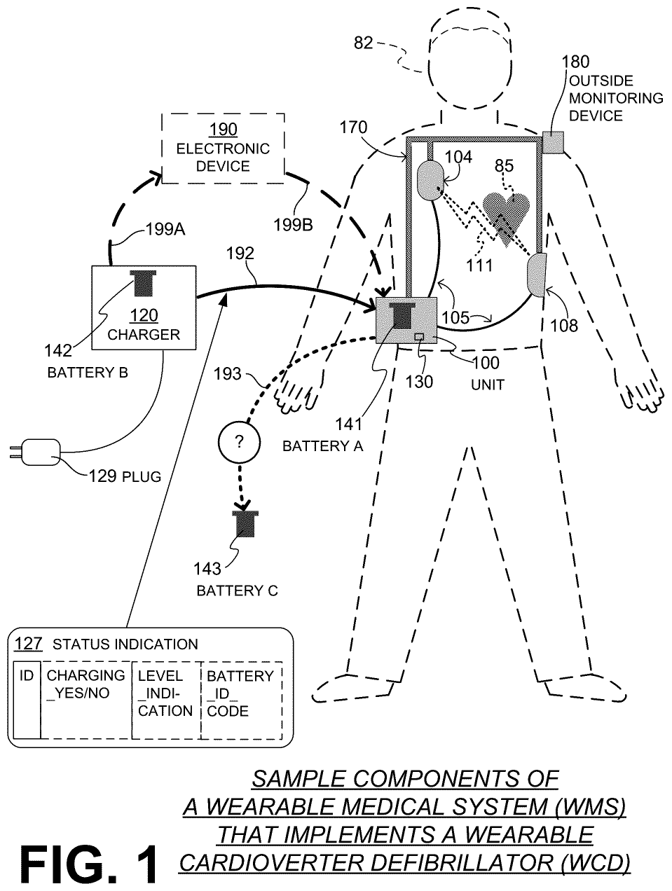

FIG. 1 depicts a patient 82. The patient 82 may also be referred to as the person 82 and/or wearer 82, since the patient 82 is wearing components of the WMS. The patient 82 is ambulatory, which means that, while wearing the wearable component(s) of the WMS, the patient 82 can walk around, be in a vehicle, and so on. In other words, the patient 82 is not necessarily bed-ridden. While the patient 82 may be considered to be also a “user” of the WMS, this definition is not exclusive to the patient 82. For instance, a user of the WMS may also be a clinician such as a doctor, nurse, emergency medical technician (EMT), or other similarly tasked and/or empowered individual or group of individuals. In some cases, a user may even be a bystander. The particular context of these and other related terms within this description should be interpreted accordingly.

A WMS that implements a WCD according to embodiments can be configured to defibrillate the patient who is wearing the designated components of the WMS. Defibrillating can be by the WMS delivering an electrical charge to the patient's body in the form of an electric shock. The electric shock can be delivered in one or more pulses.

In particular, FIG. 1 also depicts components of a WMS that implements a WCD and is made according to embodiments. One such component is a support structure 170 that is wearable by the ambulatory patient 82. Accordingly, the support structure 170 can be configured to be worn by the ambulatory patient 82 for at least several hours per day, and also during the night. That, for at least several days, and maybe even a few months. It will be understood that the support structure 170 is shown only generically in FIG. 1, and in fact partly conceptually. FIG. 1 is provided merely to illustrate concepts about the support structure 170, and is not to be construed as limiting how the support structure 170 is implemented, or how it is worn.

The support structure 170 can be implemented in many different ways. For example, it can be implemented in a single component or a combination of multiple components. In embodiments, the support structure 170 could include a vest, a half-vest, a garment, etc. In such embodiments such items can be worn similarly to analogous articles of clothing. In embodiments, the support structure 170 could include a harness, one or more belts or straps, etc. In such embodiments, such items can be worn by the patient around the torso, hips, over the shoulder, etc. In embodiments, the support structure 170 can include a container or housing, which can even be waterproof. In such embodiments, the support structure can be worn by being attached to the patient's body by adhesive material, for example as shown and described in U.S. Pat. No. 8,024,037. The support structure 170 can even be implemented as described for the support structure of US Pat. App. No. US2017/0056682, which is incorporated herein by reference. Of course, in such embodiments, the person skilled in the art will recognize that additional components of the WMS can be in the housing of a support structure instead of being attached externally to the support structure, for example as described in the US2017/0056682 document. There can be other examples.

The embodiments of FIG. 1 include a sample unit 100. In embodiments, the unit 100 is sometimes called a main electronics module. In embodiments, the unit 100 implements an external defibrillator. In embodiments, the unit 100 implements an external pacer instead of, or in addition to, an external defibrillator. In embodiments that include a pacer, the WMS may detect when the patient's heart rhythm slows down or when the patient has asystole, and the pacer may pace to increase the heart rate. In such embodiments, the WMS may pace the patient first, and hopefully not have to resort to the full intervention of defibrillation. Of course, if the patient does not respond to the pacing and their heart rhythm deteriorates further, the WMS may then later cause one or more defibrillation shocks to be delivered.

The embodiments of FIG. 1 also include sample therapy electrodes 104, 108, which are electrically coupled to unit 100 via electrode leads 105. The therapy electrodes 104, 108 are also called defibrillation electrodes or just electrodes. The therapy electrodes 104, 108 can be configured to be worn by the patient 82 in a number of ways. For instance, the unit 100 and the therapy electrodes 104, 108 can be coupled to the support structure 170, directly or indirectly. In other words, the support structure 170 can be configured to be worn by the ambulatory patient 82 so as to maintain at least one of the therapy electrodes 104, 108 on the body of the ambulatory patient 82, while the patient 82 is moving around, etc. The therapy electrodes 104, 108 can be thus maintained on the body by being attached to the skin of the patient 82, simply pressed against the skin directly or through garments, etc. In some embodiments the therapy electrodes 104, 108 are not necessarily pressed against the skin, but become biased that way upon sensing a condition that could merit intervention by the WMS. In addition, many of the components of the unit 100 can be considered coupled to the support structure 170 directly, or indirectly via at least one of the therapy electrodes 104, 108.

When the therapy electrodes 104, 108 make good electrical contact with the body of the patient 82, the unit 100 can administer, via the therapy electrodes 104, 108, a brief, strong electric pulse 111 through the body. The pulse 111 is also known as defibrillation pulse, shock, defibrillation shock, therapy, electrotherapy, therapy shock, etc. The pulse 111 is intended to go through and restart the heart 85, in an effort to save the life of the patient 82. The defibrillation pulse 111 can have an energy suitable for its purpose, such as at least 100 Joule (“J”), 200 J, 300 J, and so on. For pacer embodiments, the pulse 111 could alternately be depicting a pacing pulse. At least some of the stored electrical charge can be caused to be discharged via at least two of the therapy electrodes 104, 108 through the ambulatory patient 82, so as to deliver to the ambulatory patient 82 a pacing sequence of pacing pulses. The pacing pulses may be periodic, and thus define a pacing period and the pacing rate. There is no requirement, however, that the pacing pulses be exactly periodic. A pacing pulse can have an energy suitable for its purpose, such as at most 10 J, 5 J, usually about 2 J, and so on. The pacer therefore is delivering current to the heart to start a heartbeat. In either case, the pulse 111 has a waveform suitable for this purpose.

A prior art defibrillator typically decides whether to defibrillate or not based on an ECG signal of the patient. However, the unit 100 may initiate defibrillation, or hold-off defibrillation, based on a variety of inputs, with the ECG signal merely being one of these inputs.

A WMS that implements a WCD according to embodiments can collect data about one or more parameters of the patient 82. For collecting such data, the WMS may optionally include at least an outside monitoring device 180. The device 180 is called an “outside” device because it could be provided as a standalone device, for example not within the housing of the unit 100. The device 180 can be configured to sense or monitor at least one local parameter. A local parameter can be a parameter of the patient 82, or a parameter of the WMS, or a parameter of the environment, as described later in this document.

For some of these parameters, the device 180 may include one or more sensors or transducers. Each one of such sensors can be configured to sense a parameter of the patient 82, or of the environment, and to render an input responsive to the sensed parameter. In some embodiments the input is quantitative, such as values of a sensed parameter; in other embodiments the input is qualitative, such as informing whether or not a threshold is crossed, and so on. Such inputs about the patient 82 are also called physiological inputs and patient inputs. In embodiments, a sensor can be construed more broadly, as encompassing more than one individual sensors.

Optionally, the device 180 is physically coupled to the support structure 170. In addition, the device 180 may be communicatively coupled with other components that are coupled to the support structure 170, such as with the unit 170. Such communication can be implemented by the device 180 itself having a communication module, as will be deemed applicable by a person skilled in the art in view of this description.

A WMS that implements a WCD according to embodiments preferably includes sensing electrodes, which can sense an ECG of the patient. In embodiments, the device 180 stands for such sensing electrodes. In those embodiments, the sensed parameter of the patient 82 is the ECG of the patient, the rendered input can be time values of a waveform of the ECG signal, and so on.

In embodiments, one or more of the components of the shown WMS may be customized for the patient 82. This customization may include a number of aspects. For instance, the support structure 170 can be fitted to the body of the patient 82. For another instance, baseline physiological parameters of the patient 82 can be measured for various scenarios, such as when the patient is lying down (various orientations), sitting, standing, walking, running, and so on. These baseline physiological parameters can be the heart rate of the patient 82, motion detector outputs, one for each scenario, etc. The measured values of such baseline physiological parameters can be used to customize the WMS, in order to make its diagnoses more accurate, since patients' bodies differ from one another. Of course, such parameter values can be stored in a memory of the WMS, and so on. Moreover, a programming interface can be made according to embodiments, which receives such measured values of baseline physiological parameters. Such a programming interface may input automatically these in the WMS, along with other data.

The unit 100 includes a processor 130. More about the processor 130 is described later in this document.

The WMS of FIG. 1 further includes a charger 120. While many of the components of the WMS are worn or carried by the patient 82 who may walk around, that is not so with the charger 120. Rather, the charger 120 has a plug 129 for plugging into an electrical outlet of a home or office, for receive line power from. As such, the charger 120 is typically placed at a fixed location.

The WMS of FIG. 1 further includes a first battery A 141, a second battery B 142 and a third battery C 143. Even more batteries may be included in the WMS. These batteries are also known as system batteries. In the arrangement of FIG. 1, the first battery A 141 is in the unit 100, powering its operations, providing the charge for defibrillation if needed, and so on. The second battery B 142 is in the charger 120, being recharged, so as to be ready to be swapped with the first battery A 141, when the latter nears emptying. The third battery C 143 is plugged neither in the unit 100 nor in the charger 120. It may be used as reserve, for example be kept in a remote office, or in the trunk of a car of the patient, and so on.

In embodiments, the processor 130 may know directly how much charge is stored in the first battery A 141, because they are both in the unit 100. In fact, the processor 130 may input or sample the value of that charge of the first battery A 141 periodically, such as once a minute, store the sampled value, and so on.

In addition, the processor 130 may learn how much charge is stored in the second battery B 142, if the charger 120 communicates to the unit 100, for instance according to an arrow 192. In fact, where the communication of the arrow 192 is possible, the processor 130 may be updated with a status indication 127 about an operation of the charger 120. The status indication 127 may include an ID field that identifies the charger 120, the WMS and the transmission, a CHARGING_YES/NO field as to whether it is charging, a level indicator of an amount of charge of a battery that is presently in the charger, and a BATTERY_ID_CODE of a battery that is presently in the charger, if provided. As such, the status indication 127 can be a status indication of the value of the charge of the second battery B 142 in which case it can be also called a status indication of the second battery level. It can alternately be said that the status indication 127 includes an indication of the second battery level. In regular operation, such a status indication may be provided periodically, such as once a minute. And, the processor 130 can be configured to parse the communication of the arrow 192, identify the fields, store values from them in a memory of the unit, and on.

The communication of the arrow 192 can be effectuated by the unit 100 and the charger 120 having suitable respective communication modules, which are shown in subsequent drawings. As such, the communication of the arrow 192 can be wireless using a suitable protocol, wired, and so on. The arrow 192 is shown as unidirectional from the charger 120 to the unit 100 to indicate that, as between the two devices, the unit 100 dominates and the charger 120 reports to it. However, strictly speaking, the unit 100 may also transmit data to the charger 120, such as a status query to elicit the transmission of the status indication 127 and so on.

The communication according to the arrow 192 can therefore be executed directly as described above. In particular, the unit communication module can be configured to receive the transmitted status indication directly as transmitted from the charger communication module.

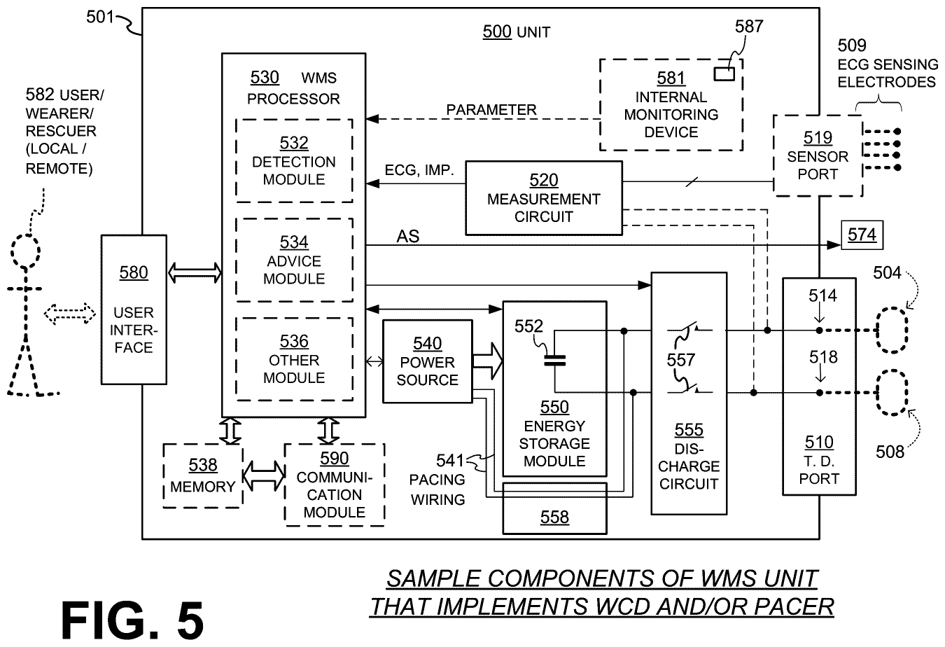

In other embodiments, the communication according to an arrow 192 can be executed indirectly, or both directly and indirectly. In indirect embodiments, the WMS also includes an electronic device 190 that is distinct from the unit 100 and from the charger 120. The electronic device 190 can be a custom device, a tablet or laptop with special software, and so on. In such embodiments, instead of the communication being effectuated according to the arrow 192, the charger 120 may communicate the status indication 127 to the electronic device 190 according to an arrow 199A, and in turn the electronic device 190 may then relay the status indication 127 to the unit 100 and thus to the processor 130. As such the electronic device 190 can be configured to receive the status indication 127 that is transmitted by the charger communication module, the electronic device 190 can be further configured to retransmit the status indication 127 it thus receives, and the unit communication module can be configured to receive the status indication that is retransmitted by the electronic device 190. Other ways are also possible. For instance, in an alternative and independent approach the unit 100 and the charger 120 may each communicate to a location in the cloud instead of directly with each other or through electronic device 190. This approach may be used instead of the previously described embodiments, so that the charger 120 sends its status and data to a database “located” in the cloud using an Internet connection. In some embodiments, the charger 120 includes a Wi-Fi module to make this Internet connection and periodically upload its status and data to the database. The unit 100 can read this data by making an Internet connection to the database. In some embodiments, the unit 100 can access this database as described below for communication module illustrated in FIG. 5. For example, the unit 100 may periodically access the database based on a schedule based on a schedule at which the charger 120 uploads its status and data. This approach may avoid requiring the unit 100 and the charger 120 being near enough to each other for their communication modules to communicate with each other, and further, may enable technicians to analyze the uploaded status and data to find ways to improve performance of the battery system.

However, while the processor 130 may know directly the first battery level, and receive the status indication 127 that includes an indication of the second battery level, there are instances where the processor 130 has no way of knowing directly the present level of the amount of charge on third battery C 143. This is a quandary indicated by an arrow 193 also having a question mark to reflect this lack of ability to know. Embodiments address this challenge, as seen later in this document.

The support structure 170 is configured to be worn by the ambulatory patient 82 so as to maintain the therapy electrodes 104, 108 on a body of the patient 82. As mentioned before, the support structure 170 can be advantageously implemented by clothing or one or more garments. Such clothing or garments do not have the function of covering a person's body as a regular clothing or garments do, but the terms “clothing” and “garment” are used in this art for certain components of the WMS intended to be worn on the human body in the same way as clothing and garments are. In fact, such clothing and garments of a WMS can be of different sizes for different patients, and even be custom-fitted around the human body. And, regular clothing can often be worn over portions or all of the support structure 170. Examples of the support structure 170 are now described.

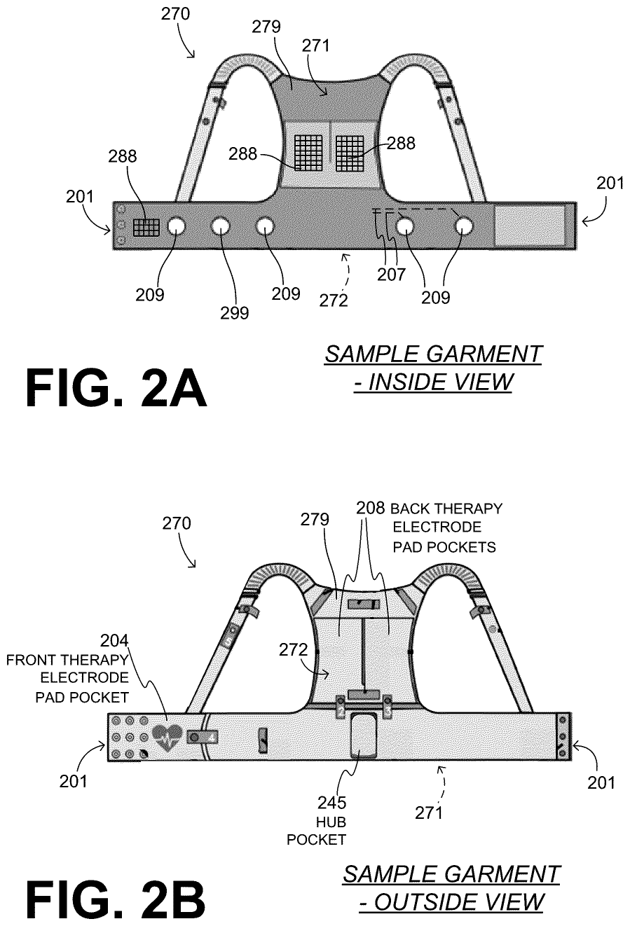

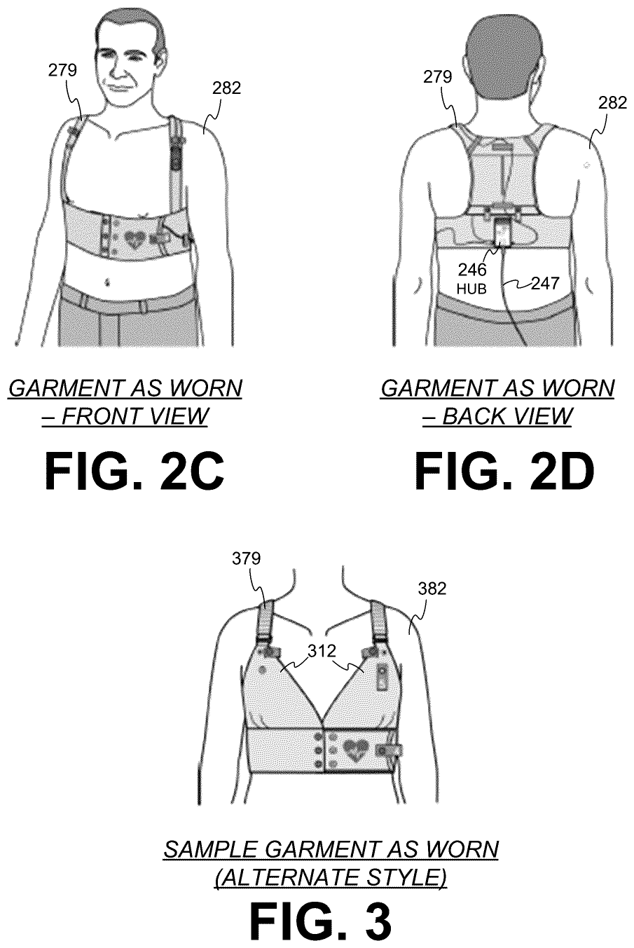

FIG. 2A shows a support structure 270 of a WMS that implements a WCD, such as the support structure 170 of FIG. 1. The support structure 270 is implemented by a vest-like wearable garment 279 that is shown flat, as if placed on a table. The inside side 271 of the garment 279 is seen as one looks at the diagram from the top, and it is the side contacting the body of the wearer when the garment 279 is worn. The outside side 272 of the garment 279 is opposite the inside side 271. To be worn, tips 201 can be brought together while surrounding the torso, and affixed to each other, either at their edges or partly overlapping. Appropriate mechanisms can hold together the tips 201, such as hooks and loops, Velcro® material, and so on.

The garment 279 can be made of suitable combinations of materials, such as fabric, linen, plastic, and so on. In places, the garment 279 can have two adjacent surfaces for defining between them pockets for the pads of the electrodes, for enclosing the leads or wires of the electrodes, and so on. Moreover, in FIG. 2A one can see meshes 288 which are the interior side of pockets accessible from the outside. The meshes can be made from flexible material such as loose netting, and so on.

ECG signals in a WMS that implements a WCD may sometimes include too much electrical noise for analyzing the ECG signal. To ameliorate the problem, multiple ECG sensing electrodes are provided in embodiments. These multiple ECG sensing electrodes define different vectors for sensing ECG signals along different ECG channels. These different ECG channels therefore present alternative options for analyzing the patient's ECG signal. The patient impedance along each ECG channel may also be sensed, and thus be part of the patient input.

In the example of FIG. 2A, multiple ECG sensing electrodes 209 are provided, which can be seen protruding from the inside surface of the garment 279. These ECG sensing electrodes 209 can be affixed to the inside surface of the garment 279, while their leads or wires 207 can be located mostly or completely within the garment 279. These ECG sensing electrodes 209 are intended to contact the skin of the person when the garment 279 is worn, and can be made from suitable material for good electrical contact. Such a material can be a metal, such as silver. An additional ECG-sensing electrode 299 may play the role of a Right Leg Drive (“RLD”) in the ECG analysis. It will be understood that “RLD” is a name for a specific ECG lead, and embodiments do not require that the electrode 299 be actually placed on the patient's right leg.

FIG. 2B shows the outside side 272 of the garment 279. One can appreciate that pockets are included that are accessible from the outside, such as a hub pocket 245. In addition a pocket 204 is provided for a front therapy electrode pad, plus two pockets 208 are provided for two back therapy electrode pads. The pads of the therapy electrodes can be placed in the pockets 204, 208, and contact the skin of the patient through the respective meshes 288 that were seen in FIG. 2A. The electrical contact can be facilitated by conductive fluid that can be deployed in the area, when the time comes for a shock.

FIG. 2C is a diagram showing a front view of how the garment 279 would be worn by a patient 282. It will be appreciated that the previously described ECG sensing electrodes 209, 299 of FIG. 2A are maintained against the body of the patient 282 from the inside side of the garment 279, and thus are not visible in FIG. 2C.

FIG. 2D is a diagram showing the back view of FIG. 2C. A hub 246 has been placed in the hub pocket 245 that is shown in FIG. 2B. A cable 247 emerges from the hub 246, which can be coupled with a unit for the system, as described later in this document.

FIGS. 2A-2D do not show any physical support for a unit such as the unit 100 of FIG. 1. In these embodiments, such a unit may be carried in a purse, on a belt, by a strap over the shoulder, or additionally by further adapting the garment 279, and so on.

FIG. 3 is a diagram showing a partial front view of another patient 382 wearing another garment 379. The garment 379 is of an alternate style than the garment 279, in that it further includes breast support receptacles 312, as was described for instance in U.S. Pat. No. 10,926,080. This style of garment may be more comfortable if the patient 382 is a woman.

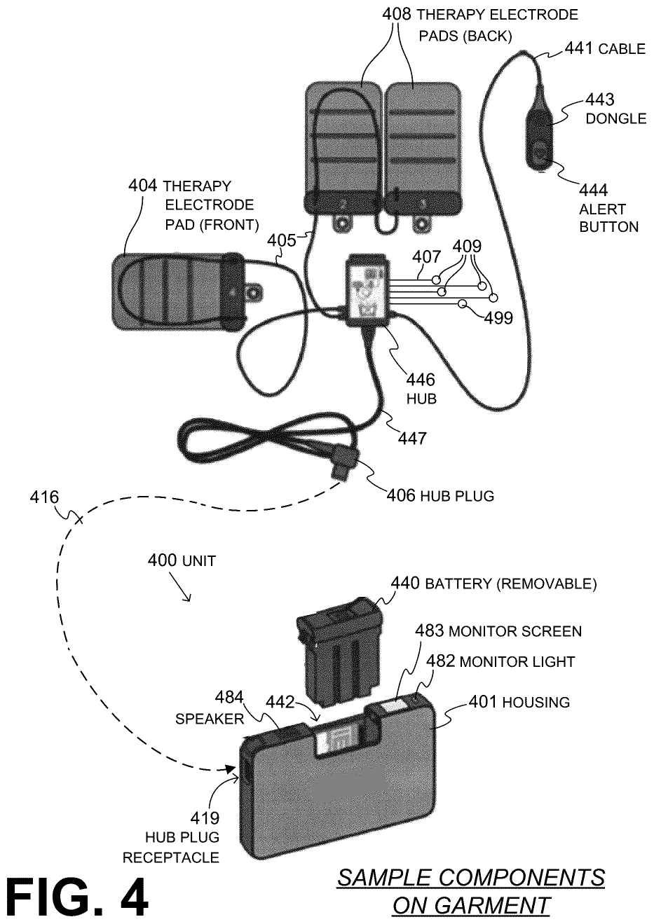

FIG. 4 shows sample electronic components that can be used with the garments 279, 379. The components of FIG. 4 include a unit 400, shown at the lower portion of FIG. 4. The unit 400 includes a housing 401, and a hub plug receptacle 419 at the housing 401.

The unit 400 includes a battery opening 442 at the housing 401. The battery opening 442 is also known as the unit receptacle, and is configured to receive a removable rechargeable battery 440. A system according to embodiments can have two or more such batteries 440, for instance as seen with the batteries 141, 142, 143 in FIG. 1. These batteries can be substantially similar to each other in shape, configured to store electrical charge, and to be rechargeable. The batteries can then be interchanged when needed, and so on.

The unit 400 also includes devices for implementing a user interface. In this example, these devices include a monitor light 482, a monitor screen 483 and a speaker 484. Additional devices may include a vibrating mechanism, and so on.

The unit 400 can implement many of the functions of the unit 100 of FIG. 1. In the embodiment of FIG. 4, however, some of the functions of the unit 100 are implemented instead by a separate hub 446, which can be connected to the unit 400. The hub 446 is smaller and lighter than the unit 400, and can accommodate multiple electrical connections to other components of FIG. 4. A cable 447, similar to the cable 247 of FIG. 2, emerges from the hub 446 and terminates in a hub plug 406. The hub plug 406 can be plugged into the hub plug receptacle 419 of the unit 400 according to an arrow 416.

ECG sensing electrodes 409, 499, plus their wires or leads 407 are further shown conceptually in FIG. 4 for completeness. The wires or leads 407 that can be configured to be coupled to the hub 446.

The components of FIG. 4 also include the therapy electrode pads 404, 408. The therapy electrode pad 404 can be inserted into the pocket 204 of FIG. 2B, while the therapy electrode pads 408 can be inserted into the pockets 208 of FIG. 2B. The shock is generated between the therapy electrode pad 404 and the therapy electrode pads 408 taken together. Indeed, the therapy electrode pads 408 are electrically connected to each other. The therapy electrode pads 404, 408, have leads 405, which can be configured to be coupled to the hub 446.

The components of FIG. 4 further include a dongle 443 with an alert button 444. The dongle 443 can be configured to be coupled to the hub 446 via a cable 441. The alert button 444 can be used by the patient to give emergency input to the WMS. For instance, the alert button 444 can be used by the patient to notify the system that the patient is actually alive and an imminent shock is not actually needed, which may otherwise happen in the event of a false positive detection of a shockable heart rhythm of the patient.

FIG. 5 shows a sample unit 500, which could be the unit 100 of FIG. 1. The unit 500 implements an external defibrillator and/or a pacer. The sample unit 500 thus combines the functions of the unit 400 and of the hub 446 of FIG. 4. The components shown in FIG. 5 can be provided in a housing 501, which may also be referred to as casing 501.

The unit 500 may include a user interface (UI) 580 for a user 582. User 582 can be the patient 82, also known as patient 582, also known as the wearer 582. Or, the user 582 can be a local rescuer at the scene, such as a bystander who might offer assistance, or a trained person. Or, the user 582 might be a remotely located trained caregiver in communication with the WMS, such as a clinician.

The user interface 580 can be made in a number of ways. The user interface 580 may include output devices, which can be visual, audible or tactile, for communicating to a user by outputting images, sounds or vibrations. Images, sounds, vibrations, and anything that can be perceived by user 582 can also be called human-perceptible indications. As such, an output device according to embodiments can be configured to output a human-perceptible indication (HPI). Such HPIs can be used to alert the patient, sound alarms that may be intended also for bystanders, and so on. There are many instances of output devices. For example, an output device can be a light that can be turned on and off, a screen to display what is sensed, detected and/or measured, and provide visual feedback to the local rescuer 582 for their resuscitation attempts, and so on. Another output device can be a speaker, which can be configured to issue voice prompts, alerts, beeps, loud alarm sounds and/or words, and so on. These can also be for bystanders, when defibrillating or just pacing, and so on. Examples of output devices were the monitor light 482, the monitor screen 483 and the speaker 484 of the unit 400 seen in FIG. 4.

The user interface 580 may further include input devices for receiving inputs from users. Such users can be the patient 82, 582, perhaps a local trained caregiver or a bystander, and so on. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock, or of pacing pulses. In particular, in some embodiments a speaker of the WMS is configured to output a warning prompt prior to an impending or planned defibrillation shock or a pacing sequence of pacing pulses being caused to be delivered, and the cancel switch is configured to be actuated by the ambulatory patient 82 in response to the warning prompt being output. In such embodiments, the impending or planned defibrillation shock or pacing sequence of the pacing pulses is not caused to be delivered. An example of a cancel switch was the alert button 444 seen in FIG. 4.

The unit 500 may include an internal monitoring device 581. The device 581 is called an “internal” device because it is incorporated within the housing 501. The monitoring device 581 can sense or monitor patient parameters such as patient physiological parameters, system parameters and/or environmental parameters, all of which can be called patient data. In other words, the internal monitoring device 581 can be complementary of, or an alternative to, the outside monitoring device 180 of FIG. 1. Allocating which of the parameters are to be monitored by which of the monitoring devices 180, 581 can be done according to design considerations. The device 581 may include one or more sensors, as also described elsewhere in this document.

Patient parameters may include patient physiological parameters. Patient physiological parameters may include, for example and without limitation, those physiological parameters that can be of any help in detecting by the WMS whether or not the patient is in need of a shock or other intervention or assistance. Patient physiological parameters may also optionally include the patient's medical history, event history and so on. Examples of such parameters include the above-described electrodes to detect the ECG, blood oxygen level, blood flow, blood pressure, blood perfusion, pulsatile change in light transmission or reflection properties of perfused tissue, heart sounds, heart wall motion, breathing sounds and pulse. Accordingly, the monitoring devices 180, 581 may include one or more sensors or transducers configured to acquire patient physiological signals. Examples of such sensors and transducers include one or more electrodes to detect ECG data, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter reflects a trend that can be detected in a monitored physiological parameter of the patient 82, 582. Such a trend can be detected by comparing values of parameters at different times over short and long terms. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above, f) respiratory function, respiratory rate, etc.; g) motion, level of activity; and so on. Once a trend is detected, it can be stored and/or reported via a communication link, along perhaps with a warning if warranted. From the report, a physician monitoring the progress of the patient 82, 582 will know about a condition that is either not improving or deteriorating.

Patient state parameters include recorded aspects of the patient 582, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed of the patient can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A WMS made according to embodiments may thus include a motion detector. In embodiments, a motion detector can be implemented within the outside monitoring device 180 or within the internal monitoring device 581. A motion detector of a WMS according to embodiments can be configured to detect a motion event. A motion event can be defined as is convenient, for example a change in posture or motion from a baseline posture or motion, etc. In such cases, a sensed patient parameter is motion. Such a motion detector can be made in many ways as is known in the art, for example by using an accelerometer and so on. In this example, a motion detector 587 is implemented within the monitoring device 581.

System parameters of a WMS can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if the monitoring device 180 or 581 includes a GPS location sensor as per the above, and if it is presumed or sensed that the patient is wearing the WMS.

The unit 500 includes a therapy delivery port 510 and a sensor port 519 in the housing 501. In contrast, in FIG. 4 these ports are located at the hub 446.

In FIG. 5, the therapy delivery port 510 can be a socket in the housing 501, or other equivalent structure. The therapy delivery port 510 includes electrical nodes 514, 518. Therapy electrodes 504, 508 are shown, which can be as the therapy electrodes 104, 108. Leads of the therapy electrodes 504, 508, such as the leads 105 of FIG. 1, can be plugged into the therapy delivery port 510, so as to make electrical contact with the nodes 514, 518, respectively. It is also possible that the therapy electrodes 504, 508 are connected continuously to the therapy delivery port 510, instead. Either way, the therapy delivery port 510 can be used for guiding, via electrodes, to the wearer at least some of the electrical charge that has been stored in an energy storage module 550 that is described more fully later in this document. When thus guided, the electric charge will cause the shock 111 to be delivered.

The sensor port 519 is also in the housing 501, and is also sometimes known as an ECG port. The sensor port 519 can be adapted for plugging in the leads of ECG sensing electrodes 509. The ECG sensing electrodes 509 can be as the ECG sensing electrodes 209. The ECG sensing electrodes 509 in this example are distinct from the therapy electrodes 504, 508. It is also possible that the sensing electrodes 509 can be connected continuously to the sensor port 519, instead. The electrodes 509 can be types of transducers that can help sense an ECG signal of the patient, e.g. a 12-lead signal, or a signal from a different number of leads, especially if they make good electrical contact with the body of the patient and in particular with the skin of the patient. As with the therapy electrodes 504, 508, the support structure can be configured to be worn by the patient 582 so as to maintain the sensing electrodes 509 on a body of the patient 582. For example, the sensing electrodes 509 can be attached to the inside of the support structure 170 for making good electrical contact with the patient, similarly with the therapy electrodes 504, 508. As such, the ECG sensing electrodes 509 are examples of sensors configured to sense a parameter, i.e. the ECG or impedance, of the ambulatory patient 82.

Optionally a WMS according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel. As such, it will not flow too far away from the location it is released. The fluid can be used for both the therapy electrodes 504, 508, and for the sensing electrodes 509.

The fluid may be initially stored in a fluid reservoir, not shown in FIG. 5. Such a fluid reservoir can be coupled to the support structure. In addition, a WMS according to embodiments further includes a fluid deploying mechanism 574. The fluid deploying mechanism 574 can be configured to cause at least some of the fluid to be released from the reservoir, and be deployed near one or both of the patient body locations to which the therapy electrodes 504, 508 are configured to be attached to the patient's body. In some embodiments, the fluid deploying mechanism 574 is activated prior to the electrical discharge responsive to receiving an activation signal AS from the processor 530, which is described more fully later in this document.

In some embodiments, the unit 500 also includes a measurement circuit 520, as one or more of its modules working together with its sensors and/or transducers. The measurement circuit 520 senses one or more electrical physiological signals of the patient from the sensor port 519, if provided. Even if the unit 500 lacks a sensor port, the measurement circuit 520 may optionally obtain physiological signals through the nodes 514, 518 instead, when the therapy electrodes 504, 508 are attached to the patient. In these cases, the input reflects an ECG measurement. The patient parameter can be an ECG, which can be sensed as a voltage difference between electrodes 504, 508. In addition, the patient parameter can be an impedance (IMP. or Z), which can be sensed between the electrodes 504, 508 and/or between the connections of the sensor port 519 considered pairwise as channels. Sensing the impedance can be useful for detecting, among other things, whether these electrodes 504, 508 and/or the sensing electrodes 509 are not making good electrical contact with the patient's body at the time. These patient physiological signals may be sensed when available. The measurement circuit 520 can then render or generate information about them as inputs, data, other signals, etc. As such, the measurement circuit 520 can be configured to render a patient input responsive to a patient parameter sensed by a sensor. In some embodiments, the measurement circuit 520 can be configured to render a patient input, such as values of an ECG signal, responsive to the ECG signal sensed by the ECG sensing electrodes 509. More strictly speaking, the information rendered by the measurement circuit 520 is output from it, but this information can be called an input because it is received as an input by a subsequent stage, device or functionality.

The unit 500 also includes a processor 530, similar to the processor 130 of FIG. 1. The processor 530 may be implemented in a number of ways. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The processor 530 may include, or have access to, a non-transitory storage medium, such as a memory 538 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

The processor 530 can be considered to have a number of modules. One such module can be a detection module 532. The detection module 532 can include a Ventricular Fibrillation (VF) detector. The patient's sensed ECG from measurement circuit 520, which can be available as inputs, data that reflect values, or values of other signals, may be used by the VF detector to determine whether the patient is experiencing VF. Detecting VF is useful, because VF typically results in SCA. The detection module 532 can also include a Ventricular Tachycardia (VT) detector for detecting VT, and so on.

Another such module in processor 530 can be an advice module 534, which generates advice for what to do. The advice can be based on outputs of the detection module 532. There can be many types of advice according to embodiments. In some embodiments, the advice is a shock/no shock determination that processor 530 can make, for example via advice module 534. The shock/no shock determination can be made by executing a stored Shock Advisory Algorithm. A Shock Advisory Algorithm can make a shock/no shock determination from one or more ECG signals that are sensed according to embodiments, and determine whether or not a shock criterion is met. The determination can be made from a rhythm analysis of the sensed ECG signal or otherwise. For example, there can be shock decisions for VF, VT, etc. As such, the processor 530 may be configured to determine whether or not a sensed parameter such as the ECG meets a defibrillation criterion.

In perfect conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in published US patent application No. US 2019/0030351 A1, and No. US 2019/0030352 A1, and which are incorporated herein by reference.

The processor 530 can include additional modules, such as other module 536, for other functions. In addition, if the internal monitoring device 581 is indeed provided, the processor 530 may receive its inputs, etc.

The unit 500 optionally further includes a memory 538, which can work together with the processor 530. The memory 538 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. The memory 538 is thus a non-transitory storage medium. The memory 538, if provided, can include programs for the processor 530, which the processor 530 may be able to read and execute. More particularly, the programs can include sets of instructions in the form of code, which the processor 530 may be able to execute upon reading. Executing is performed by physical manipulations of physical quantities, and may result in functions, operations, processes, acts, actions and/or methods to be performed, and/or the processor 530 to cause other devices or components or blocks to perform such functions, operations, processes, acts, actions and/or methods. The programs can be operational for the inherent needs of the processor 530, and can also include protocols and ways that decisions can be made by the advice module 534. In addition, the memory 538 can store prompts for the user 582, if this user is a local rescuer. Moreover, the memory 538 can store data. This data can include patient data, system data and environmental data, for example as learned by the internal monitoring device 581 and the outside monitoring device 180. The data can be stored in the memory 538 before it is transmitted out of the unit 500, or be stored there after it is received by the unit 500.

The unit 500 can optionally include a communication module 590, for establishing one or more wired or wireless communication links with other devices of other entities, such as a remote assistance center, Emergency Medical Services (EMS), and so on. The communication links can be used to transfer data and commands. The data may be patient data, event information, therapy attempted, CPR performance, system data, environmental data, and so on. For example, the communication module 590 may transmit wirelessly, e.g. on a daily basis, heart rate, respiratory rate, and other vital signs data to a server accessible over the internet, for instance as described in US 20140043149. This data can be analyzed directly by the patient's physician and can also be analyzed automatically by algorithms designed to detect a developing illness and then notify medical personnel via text, email, phone, etc. The module 590 may also include such interconnected sub-components as may be deemed necessary by a person skilled in the art, for example an antenna, portions of a processor, supporting electronics, outlet for a telephone or a network cable, etc.

The unit 500 may also include a power source 540, which is configured to provide electrical charge in the form of a current. To enable portability of the unit 500, the power source 540 typically includes a battery. Such a battery is typically implemented as a battery pack, which can be rechargeable or not. Sometimes a combination is used of rechargeable and non-rechargeable battery packs. An example of a rechargeable battery 540 was a battery 440 of FIG. 4. Other embodiments of the power source 540 can include an AC power override, for where AC power will be available, an energy-storing capacitor, and so on. Appropriate components may be included to provide for charging or replacing the power source 540. In some embodiments, the power source 540 is controlled and/or monitored by the processor 530.

The unit 500 may additionally include an energy storage module 550. The energy storage module 550 can be coupled to receive the electrical charge provided by the power source 540. The energy storage module 550 can be configured to store the electrical charge received by the power source 540. As such, the energy storage module 550 is where some electrical energy can be stored temporarily in the form of an electrical charge, when preparing it for discharge to administer a shock. In embodiments, the module 550 can be charged from the power source 540 to the desired amount of energy, for instance as controlled by the processor 530. In typical implementations, the module 550 includes a capacitor 552, which can be a single capacitor or a system of capacitors, and so on. In some embodiments, the energy storage module 550 includes a device that exhibits high power density, such as an ultracapacitor. As described above, the capacitor 552 can store the energy in the form of an electrical charge, for delivering to the patient.

As mentioned above, the patient is typically shocked when the shock criterion is met. In particular, in some embodiments the processor 530 is configured to determine from the patient input whether or not a shock criterion is met, and cause, responsive to the shock criterion being met, at least some of the electrical charge stored in the module 550 to be discharged via the therapy electrodes 104, 108 through the ambulatory patient 82 while the support structure is worn by the ambulatory patient 82 so as to deliver the shock 111 to the ambulatory patient 82. Delivering the electrical charge is also known as discharging and shocking the patient.

For causing the discharge, the unit 500 moreover includes a discharge circuit 555. When the decision is to shock, the processor 530 can be configured to control the discharge circuit 555 to discharge through the patient at least some of all of the electrical charge stored in the energy storage module 550, especially in a desired waveform. When the decision is to merely pace, i.e., to deliver pacing pulses, the processor 530 can be configured to cause control the discharge circuit 555 to discharge through the patient at least some of the electrical charge provided by the power source 540. Since pacing requires lesser charge and/or energy than a defibrillation shock, in some embodiments pacing wiring 541 is provided from the power source 540 to the discharge circuit 555. The pacing wiring 541 is shown as two wires that bypass the energy storage module 550, and only go through a current-supplying circuit 558. As such, the energy for the pacing is provided by the power source 540 either via the pacing wiring 541, or through the energy storage module 550. And, in some embodiments where only a pacer is provided, the energy storage module 550 may not be needed if enough pacing current can be provided from the power source 540. Either way, discharging can be to the nodes 514, 518, and from there to the therapy electrodes 504, 508, so as to cause a shock to be delivered to the patient. The circuit 555 can include one or more switches 557. The switches 557 can be made in a number of ways, such as by an H-bridge, and so on. In some embodiments, different ones of the switches 557 may be used for a discharge where a defibrillation shock is caused to be delivered, than for a discharge where the much weaker pacing pulses are caused to be delivered. The circuit 555 could also be thus controlled via the processor 530, and/or the user interface 580. As such, the processor 530 may be configured to, responsive to the sensed parameter meeting the defibrillation criterion, cause the capacitor 552 to receive charge from the battery 540 that at the time is powering the unit 100, 500 by being received in the unit receptacle. The processor 530 may be further configured to then to discharge the charge received in the capacitor 552 via the therapy electrodes 104, 108, 504, 508 through a body of the ambulatory patient 82, 582.

The pacing capability can be implemented in a number of ways. ECG sensing may be done in the processor, as mentioned elsewhere in this document, or separately, for demand or synchronous pacing. In some embodiments, however, pacing can be asynchronous. Pacing can be software controlled, e.g., by managing the defibrillation path, or a separate pacing therapy circuit (not shown) could be included, which can receive the ECG sensing, via the circuit 520 or otherwise.

A time waveform of the discharge may be controlled by thus controlling discharge circuit 555. The amount of energy of the discharge can be controlled by how much energy storage module has been charged, and also by how long the discharge circuit 555 is controlled to remain open.

In embodiments, the shock/no shock decision, e.g. the defibrillation criterion, can be made from the patient's heart rate and/or the QRS width of the patient's ECG complexes in the patient's ECG signal. Other parameters may also be used, such as information from a patient impedance signal (Z), information from a motion detection signal (MDET) that may evidence a motion of the patient, and so on. Of course, it is desired to measure these parameters as accurately as possible.

The unit 500 can optionally include other components.

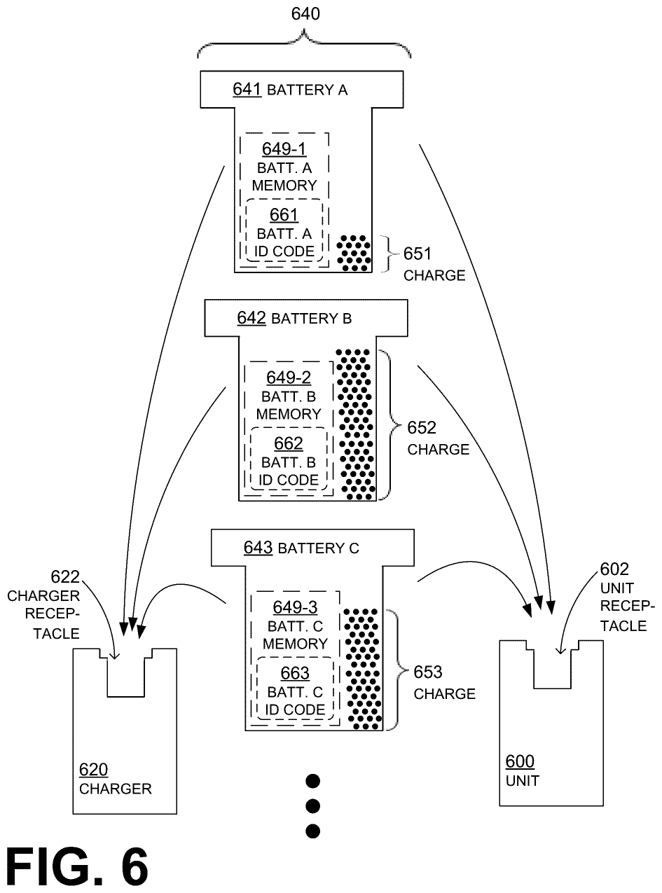

FIG. 6 is a diagram of sample components according to embodiments. A unit 600 can be as the units 100, 500. A charger 620 can be as the charger 120. A set 640 of batteries includes at least a first battery A 641, a second battery B 642, a third battery C 643, and possibly more according to the dot-dot-dot. These batteries are substantially similar to each other in shape, so they can be interchanged.

The unit 600 has a unit receptacle 602 that is configured to receive therein the first, the second, or the third battery of the set 640. The unit 600 can be powered at least by the particular one of these batteries when the particular battery is received in the unit receptacle 602. The unit 600 also has other components, for instance a clock configured to provide time inputs, a motion detector configured to provide motion detection inputs, a location indicator configured to provide a location input, and so on.

The charger 620 has a charger receptacle 622 that configured to receive therein the first, the second, or the third battery, of the set 640. The charger 620 can be configured to recharge a certain one of these batteries when the certain battery is received in the charger receptacle 622. For instance, the charger 620 can be configured to recharge the second battery 642 B when the second battery 642 B is received in the charger receptacle 622.

It will be appreciated that, in FIG. 6, the batteries of the set 640 are drawn in artificially larger scale than the unit 600 and its unit receptacle 602, and the charger 620 and its charger receptacle 622, but that is for reasons of explanation only.

In the example of FIG. 6, the batteries of set 640 are at a time instant when they have various amounts of charge. In particular, the first battery A 641 has charge 651, the second battery B 642 has charge 652, and the third battery C 643 has charge 653. As such, the battery level of the first battery A 641 is about 25%, the battery level of the second battery B 642 is 100% (full charge), and the battery level of the third battery C 643 is about 70%. It will be appreciated that the history of use of these batteries is what causes the battery levels to vary; for instance, the first battery A 641 may have been in service in the unit 600 for a long time, the second battery B 642 may have been recharged in the charger 620 and waiting to be put into service, while the third battery C 643 may have been in a remote place, not being used, and losing its charge but at a much slower rate than if it had been in service.

In the example of FIG. 6, the batteries of set 640 have ID codes, although that is not necessary. In particular, the first battery A 641 has a battery A ID code 661, the second battery B 642 has a battery B ID code 662, and the third battery C 643 has a battery C ID code 663. These ID codes are useful for tracking their history, even if only two batteries are provided with a WMS. These ID codes are even more useful when more than two batteries are provided, so that the unplugged one(s) of the batteries can be identified in some embodiments, as seen later in this document. These ID codes and suitable programming may even permit the user to nickname the individual batteries, e.g. per their intended use. For instance one battery could be nicknamed “RESERVE FOR OFFICE”, another be nicknamed “RESERVE FOR TRUNK” and so on. These nicknames can be stored at least on the processor of the unit 600, and be used in the reminder prompt that is described later in this document.

These ID codes may be implemented in a number of ways, so that such an ID code can be read when its battery is inserted in one of the receptacles 602, 622. One such way is with a chip that simply yields the ID code by electrical contacts or wirelessly, such as by RFID tags, Near Field Chip technology and so on.

Another such way is for the batteries to have respective memories that store the ID codes. These memories can be read by processors of either the unit 600 or the charger 620, or of additional processors on the batteries themselves (not shown). Sample such memories are shown in FIG. 6. In particular, the first battery A 641 has a battery A memory 649-1, the second battery B 642 has a battery B memory 649-2, and the third battery C 643 has a battery C memory 649-3.

When such memories are used, additional data can be written on them for later research, such as history of use, and so on. Moreover, the use of such memories on the batteries, permits any nicknames to be stored on the batteries themselves, instead of in a battery state table.

In embodiments, a WMS implements a WCD that may prompt the user if it perceives that the charger is not charging a swapped-out system battery. Examples are now described.

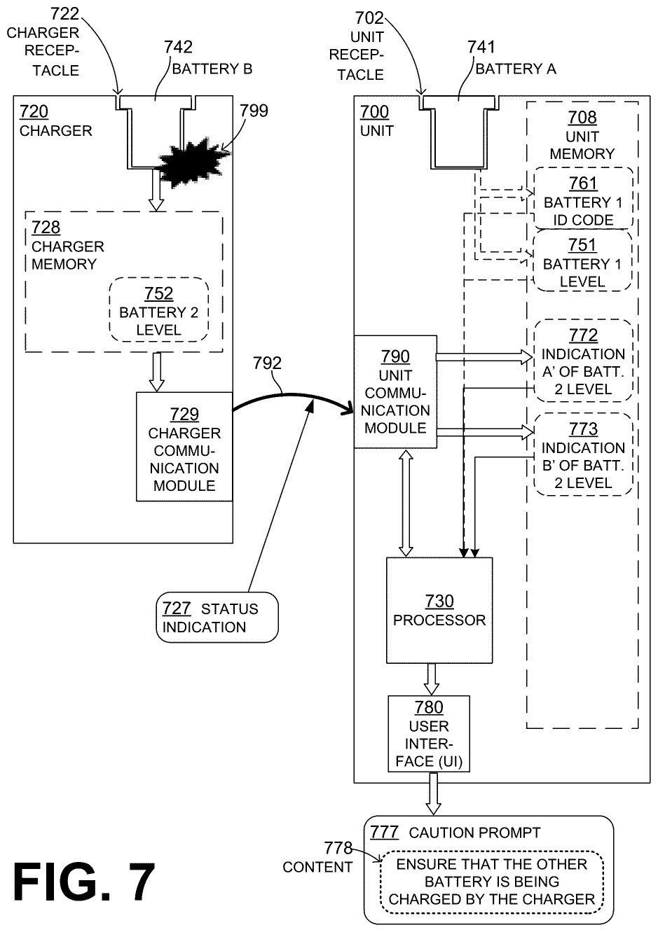

FIG. 7 is a diagram showing sample components of a WMS that implements a WCD. A unit 700 can be as described for the units 100, 500, 600. The unit 700 has a unit receptacle 702. A charger 720 can be as described for the chargers 120, 620. The charger 720 has a charger receptacle 722. A first battery A 741 and a second battery B 742 can be as described for the batteries of the set 640. In this example, the battery A 741 is properly received in the unit receptacle 702.

The battery B 742 is intended to be received in the charger receptacle 722, but it is not necessarily so, as shown by a negation icon 799. The negation of proper charging indicated by the icon 799 may happen in a number of different scenarios. For instance the patient may have received a low-charge prompt to swap the batteries, from the charger 720 to the unit 700 and vice versa. Accordingly, the patient may have properly inserted the battery A 741 in the unit receptacle 702 as shown, but may have neglected to insert the battery B 742 in the charger receptacle 722. Or, he may have inserted it, but not completely. Or, he may have inserted it, but someone such as a child may have innocently tampered with it. Or, he may have inserted it, but then line power had been lost to the outlet that was supplying the charger shortly thereafter. In the latter case, upon learning this, the patient may want to start looking for a third, reserve battery that is already substantially charged.

In FIG. 7, the charger 720 also has a charger communication module 729, and the unit 700 also has a unit communication module 790. Between them, they can execute a communication according to an arrow 792, which can be as described for the communication according to the arrow 192. In particular, the charger communication module 729 can be configured to transmit a status indication 727 about an operation of the charger 720, and the unit communication module 790 can be configured to receive the transmitted status indication 727.

The status indication 727 can be as described for the status indication 127. In some embodiments, the charger 720 further has a charger memory 728. The charger memory 728 can be configured to store a second battery level, such as battery 2 level 752 of an amount of charge stored in the battery B 742, when the battery B 742 is properly received in the charger receptacle 722, that is, before the negation of the icon 799 takes effect. In any embodiment, the charger memory 728 need not be large or standalone—in fact it can be a few memory registers of a processor in the charger. After the negation 799 takes effect, the second battery level 752 may remain stored. In some embodiments, the status indication 727 may also include an indication of the second battery level 752 that is stored in the charger memory 728.

The unit 700 also has a processor 730, which can be as described for the processors 130, 530. The unit 700 optionally also has a unit memory 708, which can be as described for the memory 538. The unit memory 708 can be configured to store data as needed, for instance a battery 1 id code 761, a battery 1 level 751, and indications of the second battery level received at different times. Two sample such indications are shown, namely indication A of battery 2 level 772 and indication B of battery 2 level 773.

The unit 700 also has a user interface (UI) 780, which can be as described for the UI 580 and/or the monitor screen 483 and/or the speaker 484. In embodiments, the processor 730 can cause the UI 780 to output a caution prompt 777 that has a content 778 to ensure that the other battery or the second battery B 742 is being charged by the charger 720. The content 778 can be delivered visually as a screen indication on a screen, as a sound prompt from a speaker, also combined with a vibration or another sound to attract the attention of the wearer, or any combination of the above.

In such embodiments, the processor 730 can be configured to and may detect whether the unit communication module 790 has received, within a time period, the transmitted status indication that the charger communication module 729 is expected to have transmitted. The processor 730 can be further configured to and may determine, from so detecting, whether or not a charger-not-charging condition is met. It may then cause, responsive to the charger-not-charging condition being met, the UI 780 to output the caution prompt 777. This may be further subject to a validation criterion being met, as described later in this document.

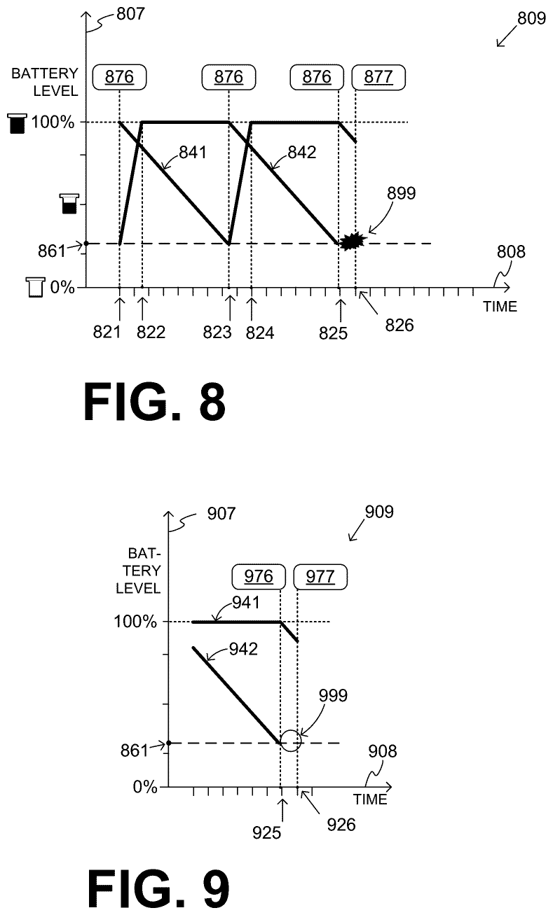

FIG. 8 is a timing diagram 809 showing time evolutions of battery levels of the batteries in the embodiments of FIG. 7, for sample scenarios.

The timing diagram 809 uses a vertical axis 807 for battery levels and a horizontal time axis 808. The time evolution of the battery level of the battery A 741 is shown as a broken dark line 841, and the time evolution of the battery level of the battery B 742 is shown as a broken dark line 842. By “broken” here it is meant that the line may have many segments, and the line may change direction abruptly from one segment to the next, as the condition and/or the environment of the battery changes.

On the vertical axis 807, the value of 100% shows when any of these batteries has been charged fully, by being in the charger 720. A value 861 indicates a certain low-charge threshold. In embodiments, when any of these batteries, while being in the unit 700, has lost so much charge as to reach the value 861, a low-charge prompt 876 is caused to be output with the content 778.

In the timing diagram 809, the low-charge prompt 876 is caused to be output at times 821, 823 and 825. In this scenario, the batteries were swapped shortly after the low-charge prompt 876 was caused to be output. As per the line 842, at a time 822 the second battery, having been in the charger 720 for a few hours, has been charged from the level of the value 861 to 100% full; and it will remain there until swapped, at time 823 responsive to the low-charge prompt 876. Similarly, as per the line 841, at times 822 824 the first battery A 741, having been in the charger 720 for a few hours, has been charged from the level of the value 861 to full; it will remain there until swapped, at times 823 and 825.

In the example of FIG. 8, there is a negation of proper charging 899 at time 826, and the caution prompt 877 is caused to be output. By that time, line 841 has decreased some, by being in the unit 700. An example is now described.

FIG. 9 is a timing diagram 909 similar to the timing diagram 809, having a vertical axis 907 for battery levels and a horizontal time axis 908. The time evolution of the battery level of the battery A 741 is shown as a broken dark line 941, and the time evolution of the battery level of the battery B 742 is shown as a dark line 942.

At time 925, the line 942 has reached the level of the value 861 and, therefore, the regular low-charge prompt 976 is caused to be output, and the batteries are swapped. Line 941 starts decreasing, because the first battery is now in service in the unit 700. However, at time 926 there is a negation of proper charging 999, and a caution prompt 977 is caused to be output. In some embodiments, the charger-not-charging condition is met responsive to detecting that the unit communication module 790 has not received the transmitted status indication within the time period. As such, the negation 999 in this example can be from receiving no status indication 127 within a time period, or receiving one where the CHARGING_YES/NO field is shown as “NO”, and no indication of battery level is included. This may happen, for instance, if the second battery B 742 is not inserted in the charger receptacle 722, or inserted but not properly.

In some embodiments, the charger further has the aforementioned charger memory 728 that is configured to store a second battery level 752 of an amount of charge stored in the second battery B 742 when the second battery B 742 is received in the charger receptacle 722, and the status indication includes an indication of the second battery level 752 that is stored in the charger memory 728. When transmitted via the communication of the arrow 792, it may be stored as the indication 772. Later, the charger communication module 729 can be further configured to transmit, via the communication of the arrow 792, an updated status indication that includes an updated indication of the second battery level, which can in turn be stored as the aforementioned indication 773. In such embodiments, the charger-not-charging condition can be met responsive to detecting that the updated indication 773 of the second battery level is not larger than the aforementioned indication 772 of the second battery level.

The devices and/or systems mentioned in this document may perform functions, processes, acts, operations, actions and/or methods. These functions, processes, acts, operations, actions and/or methods may be implemented by one or more devices that include logic circuitry. A single such device can be alternately called a computer, and so on. It may be a standalone device or computer, such as a general-purpose computer, or part of a device that has and/or can perform one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described below. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description may include flowcharts, display images, algorithms, and symbolic representations of program operations within at least one computer readable medium. An economy may be achieved in that a single set of flowcharts can be used to describe both programs, and also methods. So, while flowcharts describe methods in terms of boxes, they may also concurrently describe programs.

Methods are now described.

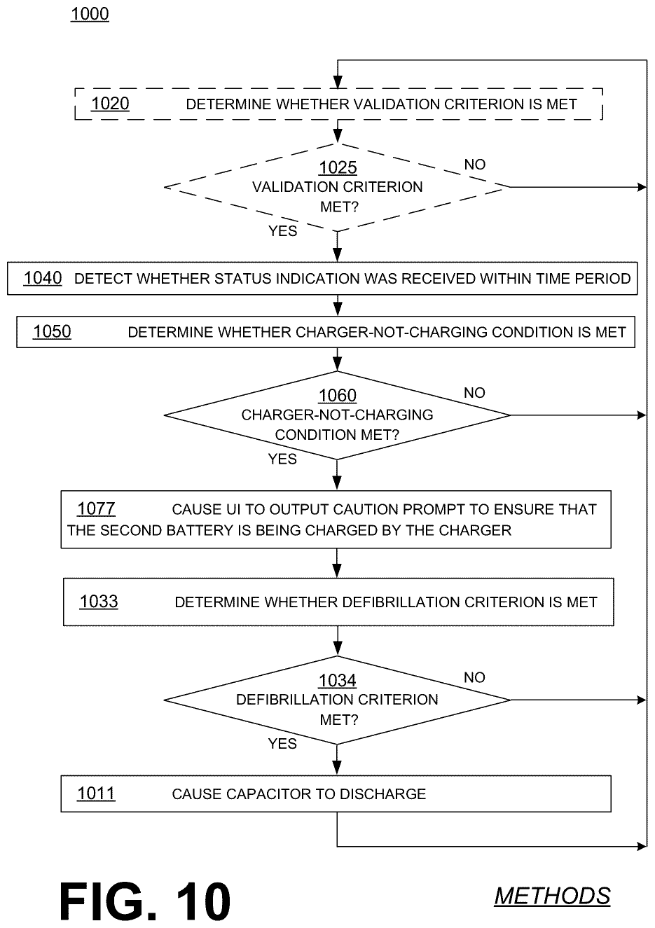

FIG. 10 shows a flowchart 1000 for describing methods according to embodiments.

According to an optional operation 1020, it may be determined whether or not a validation criterion is met. According to another, optional operation 1025, it is inquired whether or not the validation criterion is met. If the answer is NO, then execution may return to the operation 1020. As such, in embodiments the caution prompt may eventually be caused to be output only when the validation criterion is met.

If at the operation 1025 the answer is YES, then according to another operation 1040, it may be detected whether a unit communication module has received, within a time period, a transmitted status indication. According to another operation 1050, it may be determined, from the detecting of the operation 1040, whether or not a charger-not-charging condition is met.

According to another operation 1060, it is inquired whether or not the charger-not-charging condition is met. If the answer is NO, then execution may return to the operation 1020. As such, in embodiments the caution prompt is caused to be output only when the charger-not-charging condition is met.

If at the operation 1060 the answer is YES, then according to another operation 1077, the UI can be caused to output a caution prompt to ensure that the second battery is being charged by the charger.

According to another operation 1033, it may be determined whether or not a defibrillation criterion is met. The determination of the operation 1033 may be made from a sensed parameter of the patient.

According to another operation 1034, it is inquired whether or not the defibrillation criterion of the operation 1033 is met. If the answer is NO, then execution may return to the operation 1020.

If at the operation 1034 the answer is YES, then according to another operation 1011, a capacitor can be caused to receive charge from the one of the first and the second battery that is received in a unit receptacle, and then to discharge the received charge via a therapy electrode through a body of the ambulatory patient. Then execution may return to the operation 1020.

Regarding the optional validation criterion of the operation 1020, it is intended as a precaution so that the caution prompt is not caused to be output unnecessarily. A number of validation criteria may be thus employed.

In some embodiments, a processor can be further configured to determine whether or not the validation criterion is met responsive to detecting that a power-up routine has just been executed. This may indicate that it is time to check whether the battery that is removed from the unit has indeed been placed properly in the charger for recharging.

In some embodiments, a processor can be further configured to determine whether or not the validation criterion is met responsive to detecting that a different battery is received in the unit receptacle than immediately before the power-up routine. Again this may indicate that it is time to check.

Detecting that the battery is different after the power-up routine maybe accomplished by recording in the unit memory 708 one or more datapoints about the battery that is in service, data such as any one or more of its battery level, its id code, and at what time this data was captured. In particular, the processor may input a battery status datapoint of at least one of: a first level battery of an amount of charge stored in the first battery when the first battery is received in the unit receptacle, a first battery id code of the first battery when the first battery is received in the unit receptacle, and a time of the inputting or the storing the first battery level or the first battery id code, storing the inputted battery status datapoint in the unit memory, again inputting the datapoint after the power-up event, and comparing the again inputted battery status datapoint to the stored battery status datapoint.

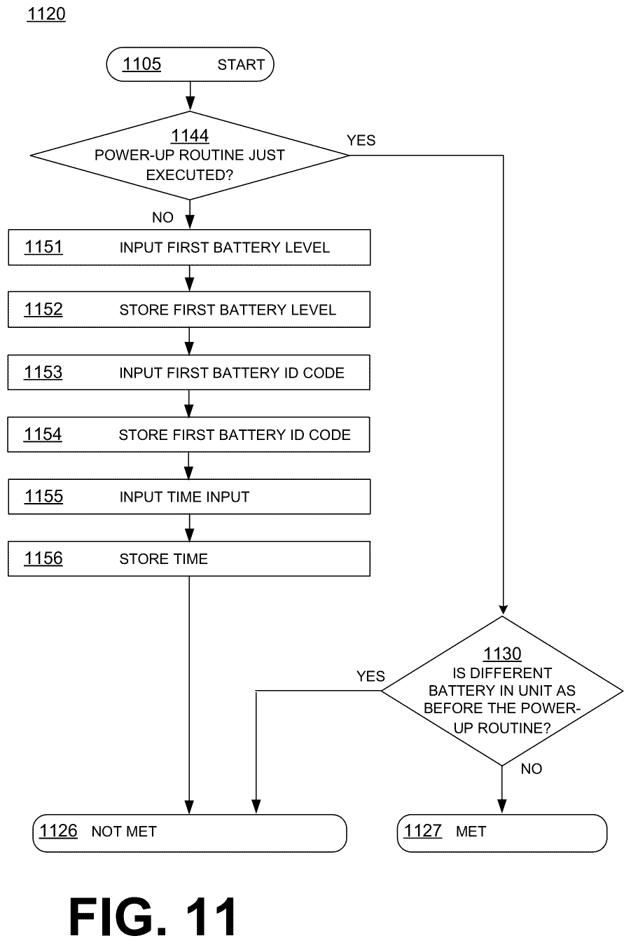

An example is now described. FIG. 11 shows a flowchart 1120 for describing a method according to embodiments for performing the operation 1020 of FIG. 10. The method of flowchart 1120 can start at a step 1105.

According to a next operation 1144, it may be determined and inquired whether or not a power-up routine has just been executed.

If the answer is YES, then according to another operation 1130, it may be inquired and determined whether or not a different battery is received in the unit receptacle than immediately before the power-up routine. The determination can be per the comparison mentioned above, optionally also using a battery state table, and so on. If the answer is NO, then it can be decided that the validation criterion is not met at a step 1127. If the answer is YES, it can be decided that the validation criterion is met at a step 1126.

If at the operation 1144 the answer is NO, then data of the present battery may be recorded to provide a future comparison.

For instance, according to an operation 1151, the first battery level of the battery A 741 may be input by the processor 730. According to a next operation 1152, the first battery level may be stored in the unit memory 708.

For another instance, according to an operation 1153, the id code of the battery A 741 may be input by the processor 730. According to a next operation 1154, the id code may be stored in the unit memory 708.

For another instance, according to an operation 1155, a time input may be input of a time at which any one of the operations 1151, 1152, 1153, 1154 was performed. According to a next operation 1156, the time input may be stored in the unit memory 708. And then it can be decided that the validation criterion is met at the step 1126.

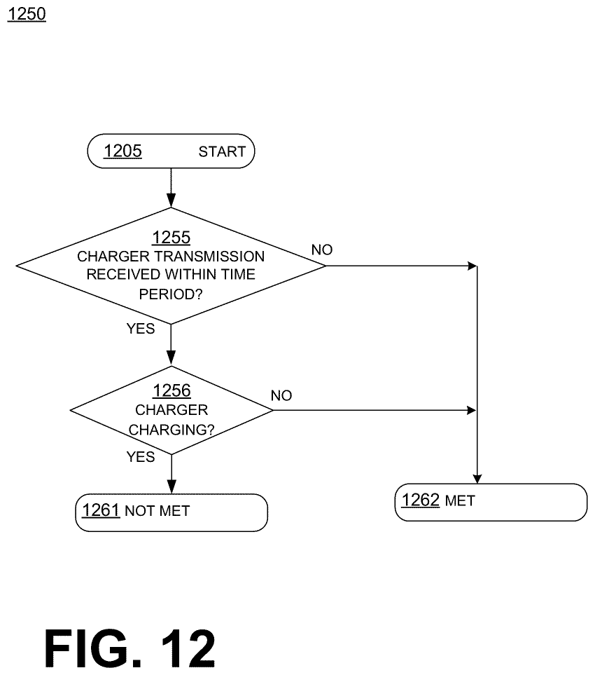

FIG. 12 shows a flowchart 1250 for describing a method according to embodiments for performing the operation 1050 of FIG. 10. The method of flowchart 1250 can start at a step 1205.

According to a next operation 1255, it may be inquired whether or not a status indication has been received, which would have been transmitted by the charger communication module. This can be answered by the operation 1040. If the answer is NO, then it can be decided that the charger-not-charging condition is met at a step 1262.

If at the operation 1255 the answer is YES, then according to a next operation 1256 it may be inquired whether or not the charger is charging. This may be performed in a number of ways, e.g. from the contents of the status indication. These may include that the charger is indeed charging the battery that is in the charger receptacle, or the most updated indication of battery level 773, which may be compared with the previous indication of battery level 772, and so on. If the answer is NO, then it can be decided that the charger-not-charging condition is met at a step 1262. If the answer is YES, then it can be decided that the charger-not-charging condition is not met at a step 1261.

In second embodiments, a WMS implements a WCD that outputs a low-charge prompt when the battery in the unit reaches a low-charge condition, but the low-charge prompt is not sent if the battery in the charger has even less charge than the battery in the unit. Instead, other measures may be taken.

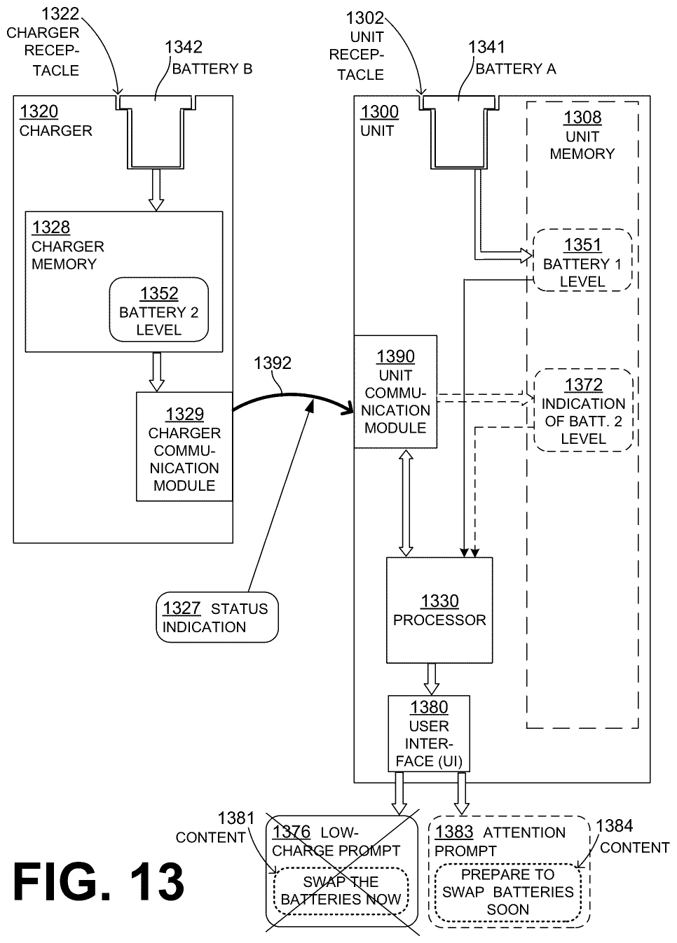

FIG. 13 is a diagram showing sample components of a WMS that implements a WCD. Many of these components can be made as described earlier in this document for similar components. In particular, a unit 1300 has a unit receptacle 1302, into which a first battery A 1341 is properly received. A charger 1320 has a charger receptacle 1322, into which a second battery B 1342 is properly received.

In some embodiments, the charger 1320 also has a charger communication module 1329, and the unit 1300 also has a unit communication module 1390. Between them, they can execute a communication according to an arrow 1392. In particular, the charger communication module 1329 can be configured to transmit a status indication 1327 about an operation of the charger 1320, and the unit communication module 1390 can be configured to receive the transmitted status indication 1327.

In some embodiments, the charger 1320 further has a charger memory 1328. The charger memory 1328 can be configured to store a second battery level, such as battery 2 level 1352 of an amount of charge stored in the battery B 1342, when the battery B 1342 is properly received in the charger receptacle 1322. In some embodiments, the status indication 1327 may also include an indication of the second battery level 1352 that is stored in the charger memory 1328.

The unit 1300 also has a processor 1330, a user interface (UI) 1380, and optionally also a unit memory 1308. If provided, the unit memory 1308 can be configured to store data as needed, for instance a battery 1 level 1351, an indication of the second battery level 1372, and so on.

In such embodiments, the processor 1330 can be configured to input a first battery level of an amount of charge stored in the first battery A 1341 when the first battery A 1341 is received in the unit receptacle, and determine, from the inputted first battery level, whether or not a low-charge condition is met. For instance, the low-charge condition may include that the inputted first battery level is below a first threshold.

The processor 1330 can be further configured to input the indication of the second battery level 1372 that is included in the received status indication 1327 and determine, from the indication of the second battery level 1372, whether or not a no-swap criterion is met. There are a number of ways to implement such a no-swap criterion. For instance, the no-swap criterion may include that the indication of the second battery level 1372 is lower than the inputted first battery level 1351. Or, the no-swap criterion may include that the indication of the second battery level 1372 is lower than the inputted first battery level 1351, but not lower than a comfort margin difference that is described later in this document.

The processor 1330 can be further configured to cause, responsive to the low-charge condition being met and responsive to the no-swap criterion not being met, the UI 1380 to output a low-charge prompt 1376 that has a content 1381 to swap the first battery A 1341 with the second battery B 1342 in the unit receptacle 1302, but not cause, responsive to the low-charge condition being met and responsive to the no-swap criterion being met, the UI 1380 to output the low-charge prompt 1376, as indicated in FIG. 13 by crossing out the low-charge prompt 1376.

Embodiments, therefore, can prevent the low-charge prompt from being output, and thus prevent the swapping when the first battery A 1341 in the unit 1300 still has more charge than the second battery B 1342 that is being charged. In such cases, other measures may be taken. For instance, the processor 1330 can be further configured to cause instead an attention prompt 1383 to be output. The attention prompt 1383 can be different from the low-charge prompt, for instance by having a content 1384 to prepare to swap the batteries soon, or other content depending on context.

For another instance, the processor 1330 can be further configured instead to cause no prompt to be output, when the no-swap criterion is met. An example of such a scenario is now described.

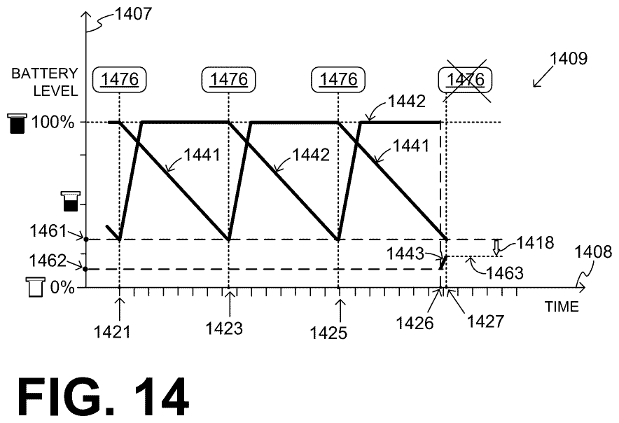

FIG. 14 is a timing diagram 1409 showing time evolutions of battery levels of the batteries in the embodiments of FIG. 13, for a specific scenario. The timing diagram 1409 uses a vertical axis 1407 for battery levels and a horizontal time axis 1408.

In this example, three batteries are involved. The time evolution of the battery level of the battery A 1341 is shown as a broken dark line 1441, the time evolution of the battery level of the battery B 1342 is shown as a broken dark line 1442, and the time evolution of the battery level of the third battery (e.g. battery C 143) is shown as a short dark line segment 1443.

At a first time 1421, the first battery A 1341 is waiting in the charger fully charged. As such, the indication of the first battery level, which the processor receives, is 100% per the line 1441. In addition, the second battery B 1342 has been in the unit receptacle, where it is normally being depleted by being in service. In particular, at that time 1421 the inputted second battery level 1442 just crossed below a first threshold 1461. Therefore, at that first time 1421 the low-charge condition is met. Notably, the no-swap criterion is not met, because the indication of the first battery level (100%) is not lower than the inputted second battery level (below the threshold 1461). Accordingly, a low-charge prompt 1476 is caused to be output, and the patient swaps the batteries.

At a subsequent time 1423, the process is repeated for the batteries in their swapped locations: the second battery B 1342 has been waiting in the charger fully charged, which is known to the processor 1330 from the indication of the second battery level (100%). In addition, the inputted first battery level 1441 just crossed below the first threshold 1461. This causes the low-charge condition to be met, while the no-swap criterion is still not met. Accordingly, the low-charge prompt 1476 is again caused to be output, and the patient swaps the batteries.

At a yet subsequent time 1425, the conditions are the same as in the first time 1421, and the process is again repeated.

In this scenario, at a yet subsequent time 1426, the patient on their own initiative has remembered the third battery, which was intended as a reserve, and had been left in a place for a long time without being used. At that time, the patient puts the third battery in the charger, while removing the second battery. At that time, the second battery B 1342 is fully charged. However, the indication of the third battery level is now line 1443. That battery level had dropped to a level 1462.

From that time on, then, the charger is charging the third battery instead of the second battery. The received status indication includes a level indication that is of the third battery (1443) instead of the second battery (1442).

Then, at a yet subsequent time 1427, the inputted first battery level 1441 just crosses below the first threshold 1461, which in turn causes the low-charge condition to be met as in the previous times 1421, 1423, 1425. At that time, however, the—now third—battery level, as learned by the processor 1330 from the status indication 1327, has risen to a value 1463, which is still lower than the first threshold 1461. Therefore, the no-swap condition is not met.

At that time 1427, the processor knows that it is time to swap the batteries, but the battery in the charger (the third battery) has even less charge than the battery in the unit. This is where the attention prompt 1383 may be caused to be transmitted.

In some embodiments, no prompt is caused to be transmitted. For instance, the no-swap criterion may include that the indication of the second—now third—battery level 1443 is lower (1462) than the inputted first battery level (1461), but not lower than a comfort margin difference. In such embodiments, a difference 1418 is determined between the level 1461 and the level 1463; if that difference is lower than the comfort margin difference, the low-charge prompt is not output at that time 1427, as indicated by the crossed-out low-charge prompt 1476. The reason is that the processor can estimate that the second battery is being charged much faster than the first battery continues to be depleted and, in a short enough time, the battery in the charger will have comfortably enough charge to be swapped out then. This is why also the no-swap criterion may include that the indication of the second battery level is within a certain value from the inputted first battery level, for instance even larger than it by 5%; it may be worth waiting for some time for the battery in the charger to be charged even more, so as to delay when the next prompt is caused to be output. Similarly, the no-swap criterion may include that the indication of the second battery level is lower than a threshold, such as a very low threshold, in the case that an empty or near empty battery was just placed in the charger.

These embodiments provide additional benefits when combined with other embodiments described elsewhere in this document.

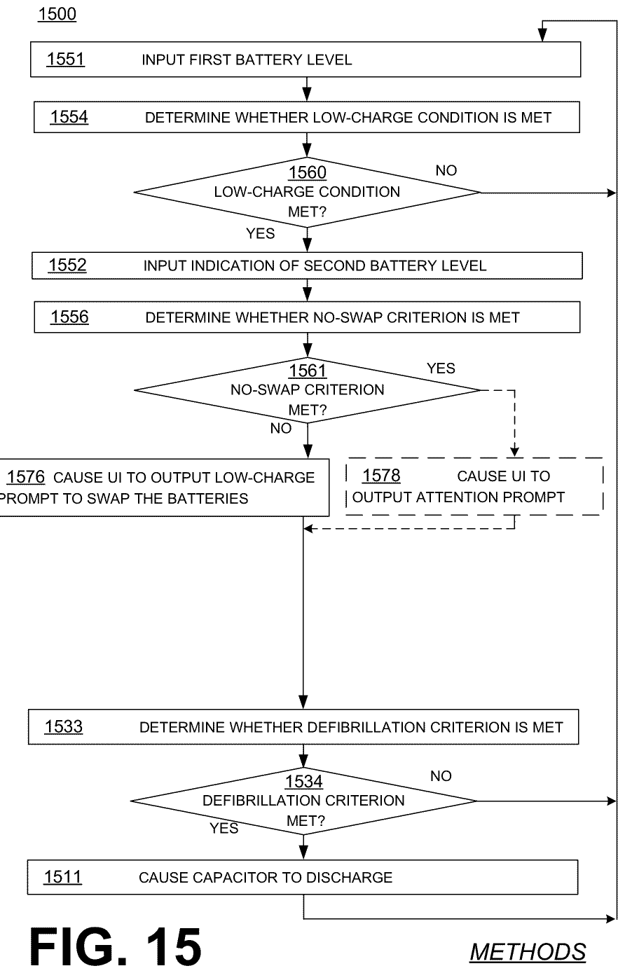

FIG. 15 shows a flowchart 1500 for describing methods according to embodiments. According to a first operation 1551, a first battery level is input.

According to another operation 1554, it may be determined whether or not a low-charge condition is met. The determination may be made from the inputted first battery level.

According to another operation 1560, it may be inquired whether or not the low-charge condition is met. The answer can be had from the operation 1554. If the answer is NO, then execution may return to the operation 1551.

If at the operation 1560 the answer is YES then, according to another operation 1552, an indication of a second battery level may be input. The indication may be included in a status indication received from a charger.

According to another operation 1556, it may be determined whether or not a no-swap criterion is met. The determination may be made from the second battery level, and possibly other data.

According to another operation 1561, it may be inquired whether or not the no-swap criterion is met. The answer can be had from the operation 1556.

If the answer is NO, then according to another operation 1576, a UI can be caused to output a low-charge prompt to swap a first battery with a second battery in a unit receptacle.

If at the operation 1561 the answer is YES, then the UI can be caused to not output the low-charge prompt. In fact, no prompt may be output. Or, according to another, optional operation 1578, an attention prompt may be caused to be output.

Additional operations 1533, 1534 and 1511 may be performed as described respectively for the operations 1033, 1034 and 1011 of FIG. 10.

In embodiments, a WMS implements a WCD that prompts, upon an adverse patient event such as charging and/or discharging the large capacitor, to swap the system battery earlier than otherwise. Sometimes this prompting is advantageously implemented in combination with the no-swap criterion. Examples are now described.

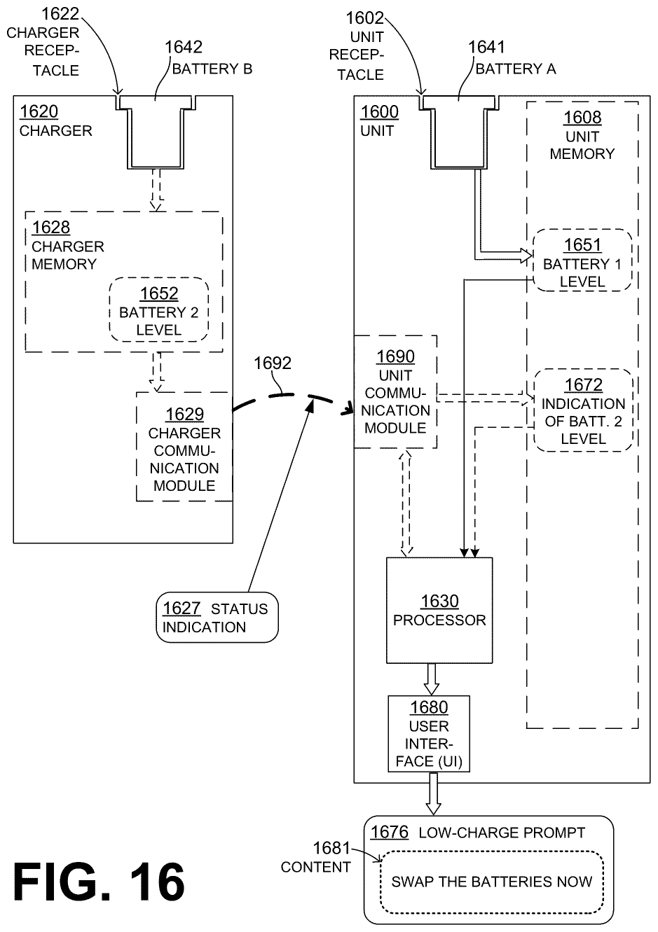

FIG. 16 is a diagram showing sample components of a WMS that implements a WCD. Many of these components can be made as described earlier in this document for similar components. In particular, a unit 1600 has a unit receptacle 1602, into which a first battery A 1641 is properly received. A charger 1620 has a charger receptacle 1622, into which a second battery B 1642 is properly received.

In some embodiments, the charger 1620 also has a charger communication module 1629, and the unit 1600 also has a unit communication module 1690. Between them, they can execute a communication according to an arrow 1692. In particular, the charger communication module 1629 can be configured to transmit a status indication 1627 about an operation of the charger 1620, and the unit communication module 1690 can be configured to receive the transmitted status indication 1627.

In some embodiments, the charger 1620 further has a charger memory 1628. The charger memory 1628 can be configured to store a second battery level, such as battery 2 level 1652, of an amount of charge stored in the battery B 1642, when the battery B 1642 is properly received in the charger receptacle 1622. In some embodiments, the status indication 1627 may also include an indication of the second battery level 1652 that is stored in the charger memory 1628.

The unit 1600 also has a processor 1630, a user interface (UI) 1680, and optionally also a unit memory 1608. If provided, the unit memory 1608 can be configured to store data as needed, for instance a battery 1 level 1651, an indication of the second battery level 1672, and so on.

In such embodiments, the processor 1630 can be configured to input a first battery level of an amount of charge stored in the first battery A 1641 when the first battery A 1641 is received in the unit receptacle 1602, and determine, from the inputted first battery level, whether a low-charge condition is met. For instance, the low-charge condition may include that the inputted first battery level is below a first threshold. The processor 1630 can be further configured to cause, responsive to the low-charge condition being met, the UI 1680 to output a low-charge prompt 1676 that has a content 1681 to swap the first battery A 1641 with the second battery B 1642 in the unit receptacle 1602.

The processor 1630 can be further configured to update the low-charge condition responsive to one of the capacitor being caused to receive the charge and the capacitor being caused to discharge the received charge. The updating may include changing the low-charge condition from a first condition to a second condition. For instance, the first condition may include that the inputted first battery level is below a first threshold, and the second condition may include that the inputted first battery level is below a second threshold instead of the first threshold, the second threshold higher than the first threshold.

The update is optionally temporary, and the previous low-charge condition may be restored afterwards. For instance, in embodiments the processor 1630 can be further configured to cancel the update of the low-charge condition, the canceling including changing the low-charge condition from the second condition back to the first condition, for instance after the no-swap criterion is no longer met.

Embodiments, therefore, can cause the swapping of the batteries to be faster than otherwise, if a lot of charge has been suddenly lost by the charging and/or discharging of the large capacitor. This is advantageous for instances where one such event is expected to be followed by a cascade of similar such events.

Note that, for some embodiments, no status indications by the charger 1620 are needed by the processor 1630. In other, optional embodiments the status indication 1627 is indeed received, and used so that the no-swap criterion can also be implemented. In particular, the processor 1630 can be further configured to input the indication of the second battery level 1672 that is included in the received status indication 1627 and determine, from the indication of the second battery level 1672, whether or not a no-swap criterion is met. In such embodiments, therefore, the processor 1630 can be configured to cause, responsive to the low-charge condition being met and responsive to the no-swap criterion not being met, the UI 1680 to output the low-charge prompt 1676, but not cause, responsive to the low-charge condition being met and responsive to the no-swap criterion being met, the UI 1680 to output the low-charge prompt 1676. In such cases, other measures may be taken. For instance, the processor 1630 can be further configured to cause instead an attention prompt to be output, such as was described previously. For another instance, the processor 1630 can be further configured instead to cause no prompt to be output, when the no-swap criterion is met.

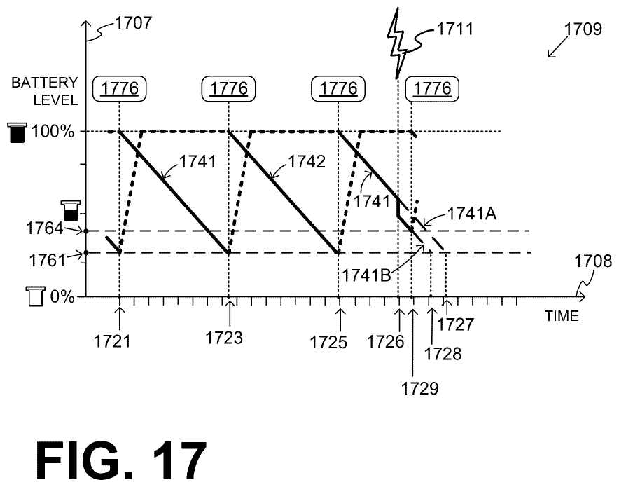

FIG. 17 is a timing diagram 1709 showing time evolutions of battery levels of the batteries in the embodiments of FIG. 16, for a specific scenario. The timing diagram 1709 uses a vertical axis 1707 for battery levels and a horizontal time axis 1708.

The time evolution of the battery level of the battery A 1641 is shown as a broken dark line 1741, and the time evolution of the battery level of the battery B 1642 is shown as a broken dark line 1742. It will be observed that, differently from previous such drawings, the lines 1741 and 1742 have segments that are solid and segments that are dotted. The solid segments of the lines 1741 and 1742 are for when their respective batteries 1641, 1642 are in the unit receptacle 1602, and thus their battery levels are directly sampled and inputted by the processor 1630. The dotted segments of the lines 1741 and 1742 are for when their respective batteries 1641, 1642 are received in the charger receptacle 1622, in which case their values, while accurate, are not learned by the processor 1630 in the embodiments where the status indication 1627 is received.

At times 1721, 1723, 1725, the evolution of these diagrams is similar to those in FIG. 14. As such, low-charge prompts 1776 have been caused to be output at those times, because that is when the battery level of the battery in the unit 1600 just crosses below the first threshold 1761.

In the scenario of FIG. 17, if no significant event happens after time 1725, the next low-charge prompt 1776 would be expected to occur at a time 1727, which is when the future projection 1741A of the line 1741 will cross again the first threshold 1761. In the scenario of FIG. 17, that is not what happens. Instead, at a subsequent time 1726 that occurs before 1727, an event 1711 takes place. The event 1711 can be charging the capacitor and/or discharging the capacitor, for instance when preparing for and/or administering defibrillation. As such, at that time 1726, the line 1741 experiences a vertical drop. Its future projection 1741B now would be expected to cross the first threshold 1761 at an earlier time 1728.

As mentioned above, in embodiments the processor 1630 can be further configured to update the low-charge condition. In the example of FIG. 17, an updated threshold 1764 is established, instead of the first threshold 1761. The second threshold 1764 is higher than the first threshold 1761. The result is that the line 1741 crosses the updated threshold 1764 at a time 1729, which is even earlier than the time 1728. At the time 1729, therefore, the low charge prompt is caused to be output.

This aggressive technique is intended to ensure that the unit 1600 is prepared with a more-ready battery for a possible cascade of such adverse patient events that may follow the event 1711, and each of which can require a lot of charge by the WMS. Again, the no-swap criterion can be used so that the aggressive technique will be implemented only when beneficial.

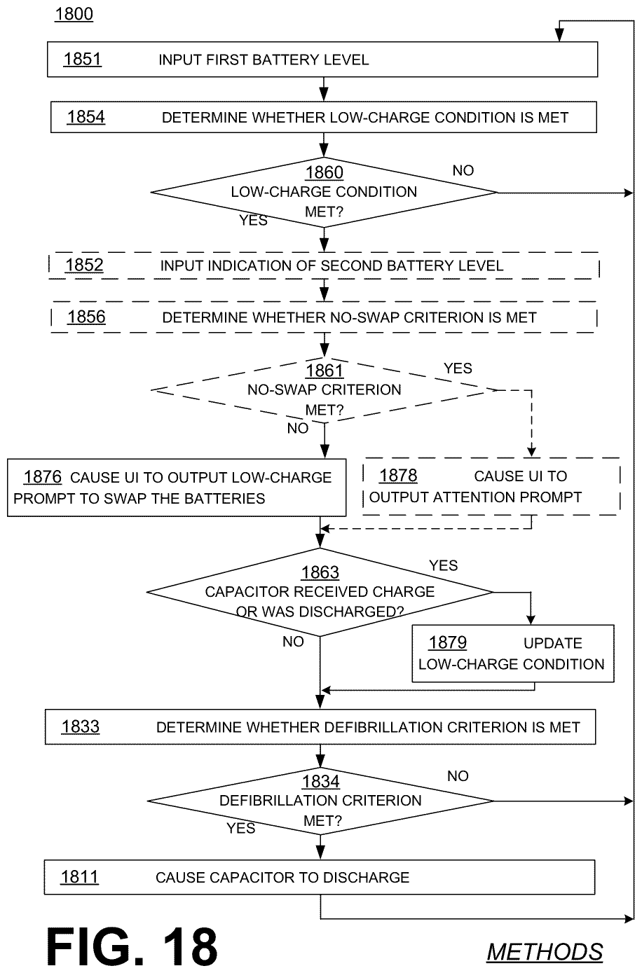

FIG. 18 shows a flowchart 1800 for describing methods according to embodiments. According to a first operation 1851, a first battery level is input.

According to another operation 1854, it may be determined whether or not a low-charge condition is met. The determination may be made from the inputted first battery level.

According to another operation 1860, it may be inquired whether or not the low-charge condition is met. The answer can be had from the operation 1854. If the answer is NO, then execution may return to the operation 1851.

If at the operation 1860 the answer is YES then, in some embodiments, the no-swap criterion is also implemented. For instance, according to another, optional operation 1852, an indication of a second battery level may be input. The indication may be included in a status indication received from a charger. According to another, optional operation 1856, it may be determined whether or not a no-swap criterion is met. The determination may be made from the second battery level, and possibly other data. Then according to another operation 1861, it may be inquired whether or not the no-swap criterion is met. The answer can be had from the operation 1856. If at the operation 1861 the answer is YES, then the UI can be caused to not output the low-charge prompt. In fact, no prompt may be output. Or, according to another, optional operation 1878, an attention prompt may be caused to be output, before execution returns to the operation 1851.

If at the operation 1861 the answer is NO, or if the no-swap criterion is not implemented then, according to another operation 1876, a UI can be caused to output a low-charge prompt to swap a first battery with a second battery in a unit receptacle.

Then, at another operation 1863, it may be inquired whether or not a capacitor has been caused to receive charge and/or a capacitor has been caused to discharge a received charge. If yes then, at another operation 1879, the low-charge condition may be updated.

Additional operations 1833, 1834 and 1811 may be performed as described respectively for the operations 1033, 1034 and 1011 of FIG. 10.

In embodiments, a WMS implements a WCD that prompts to swap the batteries at an opportune moment earlier than necessary. Examples are now described.

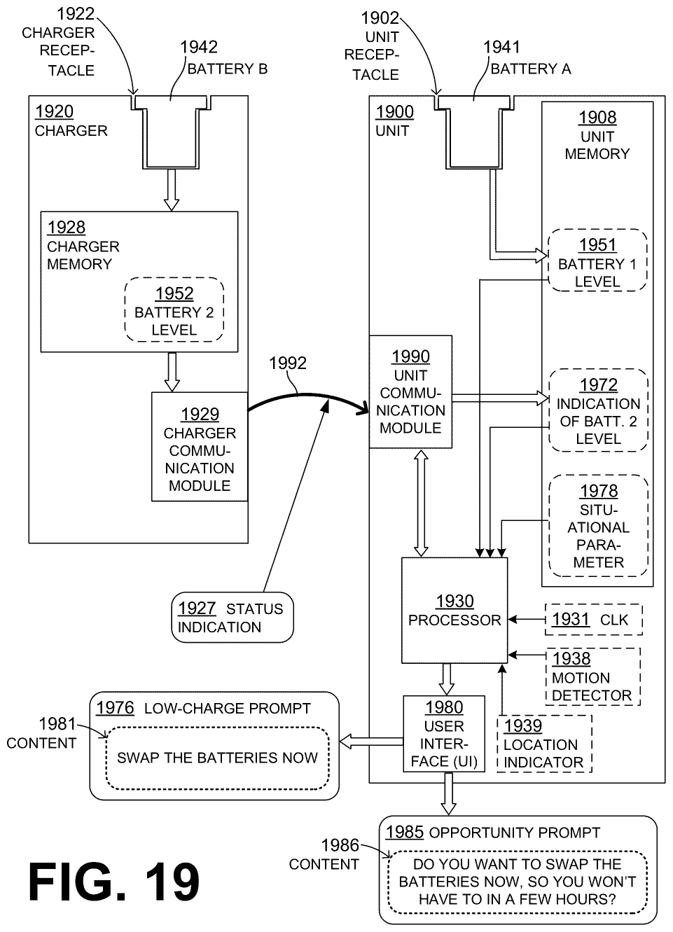

FIG. 19 is a diagram showing sample components of a WMS that implements a WCD. Many of these components can be made as described earlier in this document for similar components. In particular, a unit 1900 has a unit receptacle 1902, into which a first battery A 1941 is properly received. A charger 1920 has a charger receptacle 1922, into which a second battery B 1942 is properly received.

In some embodiments, the charger 1920 also has a charger communication module 1929, and the unit 1900 also has a unit communication module 1990. Between them, they can execute a communication according to an arrow 1992. In particular, the charger communication module 1929 can be configured to transmit a status indication 1927 about an operation of the charger 1920, and the unit communication module 1990 can be configured to receive the transmitted status indication 1927.

In some embodiments, the charger 1920 further has a charger memory 1928. The charger memory 1928 can be configured to store a second battery level, such as battery 2 level 1952 of an amount of charge stored in the battery B 1942, when the battery B 1942 is properly received in the charger receptacle 1922. In some embodiments, the status indication 1927 may also include an indication of the second battery level 1952 that is stored in the charger memory 1928.

The unit 1900 also has a processor 1930, a user interface (UI) 1980, and optionally also a clock 1931, a motion detector 1938, a location indicator 1939, and unit memory 1908. The unit memory 1908 can be configured to store data as needed, for instance a battery 1 level 1951, an indication of the second battery level 1972, a situational parameter 1978, and so on.

In such embodiments, the processor 1930 can be configured to input a first battery level of an amount of charge stored in the first battery A 1941 when the first battery A 1941 is received in the unit receptacle, store, in the unit memory 1908, the inputted first battery level 1951, and determine, from the stored first battery level, whether a low-charge condition is met. The processor 1930 can be further configured to cause, responsive to the low-charge condition being met, the UI 1980 to output a low-charge prompt like the low-charge prompt 1676.

The processor 1930 can be further configured to input the indication of the second battery level 1952 that is included in the received status indication, and store, in the unit memory 1908, the inputted indication of the second battery level 1972. Note that the indication 1972 has a different reference numeral than the second battery level 1952 itself, because they could be different. In some embodiments, they are identical.

The processor 1930 can be further configured to input a situational parameter 1978, which may be stored in the unit memory 1908. In some embodiments, the processor 1930 can be configured to also prepare the situational parameter, as is explained in more detail later in this document.

The processor 1930 can be further configured to determine, from the stored first battery level 1951, from the stored indication of the second battery level 1972, and from the situational parameter 1978, but not from an operation of the capacitor that is used for defibrillation, whether or not an opportune condition is met. Responsive to the opportune condition being met, and responsive to the low-charge condition not being met, the processor 1930 can be further configured to cause the UI 1980 to output an opportunity prompt 1985 with content 1986 to swap, in the unit receptacle 1902, the first battery A 1941 with the second battery B 1942.

Embodiments of the opportune condition are now described. The opportune condition may involve a prediction of an upcoming length of time during which it will be undesirable to bother the patient with a low-charge prompt. As will be appreciated, embodiments of the opportune condition can be implemented by suitably constructing the situational parameter 1978. Alternatively phrased, the situational parameter 1978 can be constructed such that it can be determined from it whether or not the opportune condition is met. For instance, the situational parameter 1978 may be constructed so as to involve one or more component parameters that are expressed numerically, in Boolean form, etc. As will be further appreciated, such component parameters either involve ready data, or data that may be gathered by the processor 1930 in the context of preparing such data. Such gathering can be, for example, by the processor 1930 internally querying of the clock 1931 or its time input, the motion detector 1938 or its motion detection input, the location indicator 1939 or its location input, the unit memory 1908, preparing the component parameters as statistics from the data so that the opportune condition can be evaluated, and so on.

An illustrative example of an opportune condition is that the patient is going to sleep for the evening, and it is desirable to not wake them after that for a certain number of hours to swap the batteries. As such, in some embodiments, the opportune condition includes that the stored indication of the second battery level is higher than the stored first battery level. And, in some embodiments, the situational parameter includes a stored evening time, and it is determined whether or not the opportune condition is met at the stored evening time. And, in some embodiments, the opportune condition includes that the stored indication of the second battery level is adequate for service in the unit for at least 6 hours, 8 hours, 10 hours and so on. This inclusion can be in the form of a required component parameter.

In some embodiments, the unit 1900 further includes a clock 1931 configured to provide a time input. In such embodiments, it can be determined whether or not the opportune condition is met from the time input.

In some embodiments, the WMS further includes a location indicator, which is configured to provide a location input. For instance, such a location indicator can be the location indicator 1939 in the unit 1900, or it can be outside the unit 1900. In such embodiments, it can be determined whether or not the opportune condition is met from the location input. The location input can be a Boolean value, like true/false as to whether the patient is and remains close enough to the known location of their bed. This can be combined with the time of the day to find the typical evening bedtime, and so on. Plus, artificial intelligence (AI) can be used to learn, from their habits, their usual bedtime, and so on. Of course, such values can alternately be set by the UI during customization, and so on.

In some embodiments, the WMS further includes a motion detector, which is configured to provide a motion detection input. For instance, such a motion detector can be the motion detector 1938 in the unit 1900, or it can be outside the unit 1900. In such embodiments, it can be determined whether or not the opportune condition is met from the motion detection input. For instance the motion detection input can be an accelerometer that informs about the posture of the person, i.e. whether they are standing up (vertical), lying down (horizontal), and so on. Again, this can be combined with the time of the day to find the evening bedtime, their location, and so on.

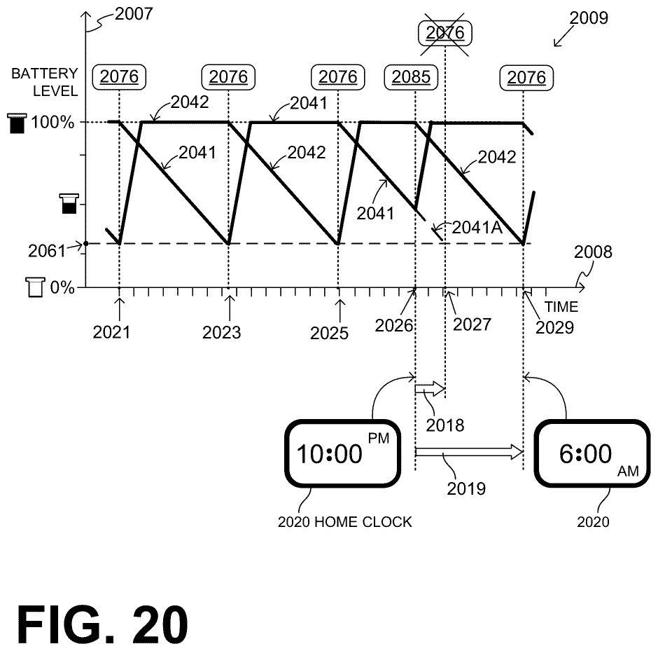

FIG. 20 is a timing diagram 2009 showing time evolutions of battery levels of the batteries in the embodiments of FIG. 19, for a specific scenario. The timing diagram 2009 uses a vertical axis 2007 for battery levels and a horizontal time axis 2008.

The time evolution of the battery level of the battery A 1941 is shown as a broken dark line 2041, and the time evolution of the battery level of the battery B 1942 is shown as a broken dark line 2042.

At times 2021, 2023, 2025, the evolution of these diagrams is similar to those in FIG. 14. As such, low-charge prompts 2076 have been caused to be output at those times, because that is when the battery level of the battery in the unit 1900 just crosses below the first threshold 2061.

In the scenario of FIG. 20, if no significant event happens after time 2025, the next low-charge prompt 2076 would be expected to occur at a time 2027, which is when the future projection 2041A of the line 2041 will cross again the first threshold 2061. In the scenario of FIG. 20, that is not what happens. Instead, at a subsequent time 2026 that occurs before 2027, the opportune condition is met, while the low-charge condition is not met. As such, at the time 2026 the UI 1980 causes to be output an opportunity prompt 2085. The batteries are then swapped again and, therefore, at time 2027 the low-charge condition is not met at the time 2027. As such, a low-charge prompt 2076 is not caused to be output at that time 2027, as indicated by a crossed-out prompt 2076. Rather, the next time that the low-charge condition is met occurs at a much later time 2029, at which time a low-charge prompt 2076 is caused to be output.

What this embodiment accomplished is that, at time 2026, the next low-charge prompt 2076, instead of occurring a time duration 2018 later, it occurred a time duration 2019 later. This accommodated the patient by not distracting them during the time duration 2019 with a low-charge prompt.

The benefit of such embodiments can be valuable for the patient if the time duration 2019 is the night, and in particular if the time 2026 is the patient's evening bed time. To narrowly illustrate that benefit by focusing only around the time duration 2019, two instances a home clock 2020 have been added, one showing the time 2026 as 10 pm, and the other showing the time 2029 as 6 am. The patient got to sleep through this night without interruption. This example with the home clock 2020 and the 8-hour interval, however, is limited to the neighborhood of the time duration 2019 should not be extrapolated in time for determining as to what might have happened at earlier times in this diagram, or how long the batteries actually last or how long the batteries actually require to be charged.

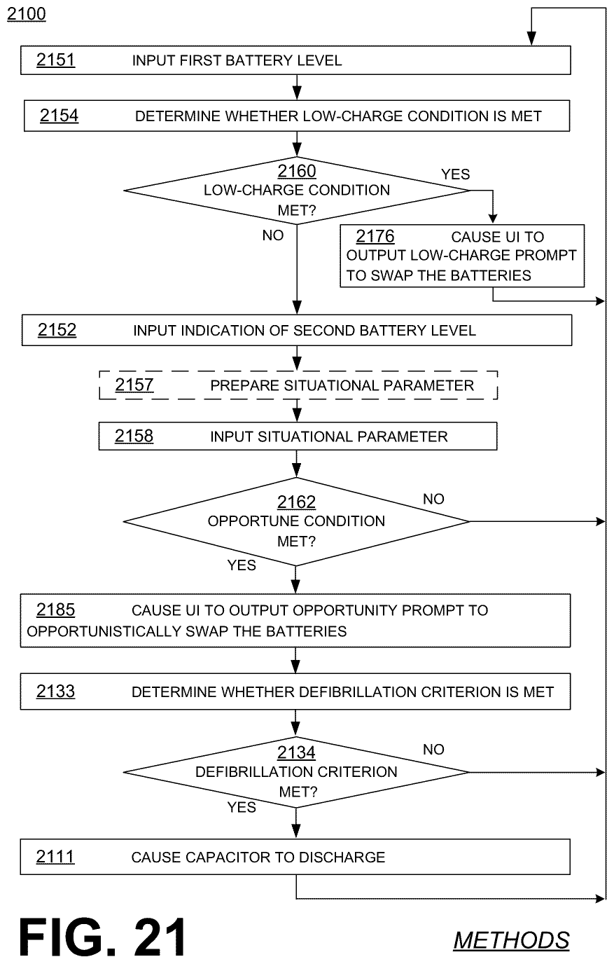

FIG. 21 shows a flowchart 2100 for describing methods according to embodiments. According to a first operation 2151, a first battery level is input.

According to another operation 2154, it may be determined whether or not a low-charge condition is met. The determination may be made from the inputted first battery level.

According to another operation 2160, it may be inquired whether or not the low-charge condition is met. The answer can be had from the operation 2154. If the answer is YES, then another, operation 2176 a UI can be caused to output a low-charge prompt, and then execution may return to the operation 2151.

If at the operation 2160 the answer is YES then, according to another operation 2152, an indication of a second battery level may be input. The indication may be included in a status indication received from a charger.

According to another, optional operation 2157, a situational parameter may be prepared. For instance, components of it may be prepared.

According to another operation 2158, a situational parameter may be input. For instance, it can be the situational parameter prepared at the operation 2157.

According to another operation 2162, it may be determined whether or not an opportune condition is met. If the answer is NO, then execution may return to the operation 2151.

If at the operation 2162 the answer is YES then, according to another operation 2185, a UI can be caused to output an opportunity prompt.

Additional operations 2133, 2134 and 2111 may be performed as described respectively for the operations 1033, 1034 and 1011 of FIG. 10.

In embodiments, a WMS implements a WCD that prompts to remind the user to fully charge also a third battery that may have been unplugged for a long time and has therefore lost some of its charge. Sometimes this prompting is advantageously implemented in combination with the no-swap criterion. Examples are now described.

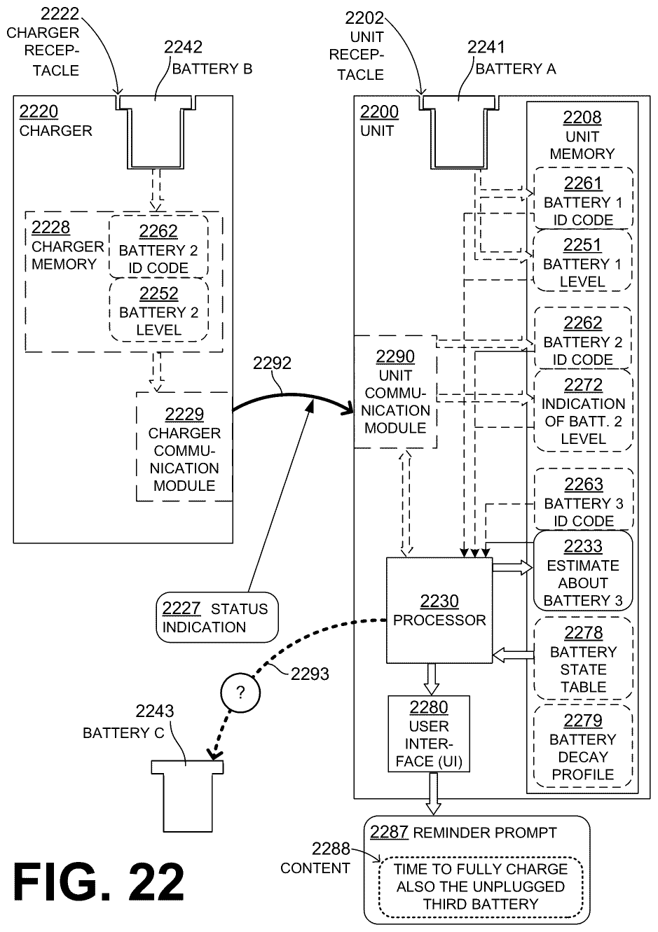

FIG. 22 is a diagram showing sample components of a WMS that implements a WCD. Many of these components can be made as described earlier in this document for similar components. In particular, a unit 2200 has a unit receptacle 2202, into which a first battery A 2241 is properly received. A charger 2220 has a charger receptacle 2222, into which a second battery B 2242 is properly received. In addition, the WMS has a third battery C 2243, which the patient may use as a reserve battery, in case they need to and they are far away from the charger 2220 at the time a swap is needed. As such, the battery C 2243 is presently not received either in the unit receptacle 2202 or in the charger receptacle 2222.

In some of these embodiments, the system batteries have ID codes as mentioned above. In particular, the first battery A 2241 has a battery 1 ID code 2261, the second battery B 2242 has a battery 2 ID code 2262, and the third battery C 2243 has a battery 3 ID code 2263.

In some embodiments, the charger 2220 also has a charger communication module 2229, and the unit 2200 also has a unit communication module 2290. Between them, they can execute a communication according to an arrow 2292. In particular, the charger communication module 2229 can be configured to transmit a status indication 2227 about an operation of the charger 2220, and the unit communication module 2290 can be configured to receive the transmitted status indication 2227.

In some embodiments, the charger 2220 further has a charger memory 2228. The charger memory 2228 can be configured to store a second battery level, such as battery 2 level 2252 of an amount of charge stored in the battery B 2242, when the battery B 2242 is properly received in the charger receptacle 2222. In some embodiments, the status indication 2227 may also include an indication of the second battery level 2252 that is stored in the charger memory 2228.

The unit 2200 also has a processor 2230, and a user interface (UI) 2280. Since the third battery C 2243 is plugged neither in the unit receptacle 2202 nor in the charger receptacle 2222, the processor 2230 has no way of knowing directly the present level of the amount of its charge, which is a quandary indicated by an arrow 2293 also having a question mark. For that, it may generate instead an estimate, as described below.

The unit 2200 also has a unit memory 2208, which can be configured to store data as needed. Such data may include instance the battery 1 ID code 2261, a battery 1 level 2251, the battery 2 ID code 2262, an indication of the second battery level 2272, the battery 3 ID code 2263, an estimate 2233 of an amount of charge remaining stored in the third battery C 2243, a battery state table 2278, a battery decay profile 2279, and so on.

In embodiments, the processor 2230 is configured to identify the third battery C 2243 as unplugged, responsive to determining that the third battery C 2243 is presently not received either in the unit receptacle or in the charger receptacle. This can be performed in a number of ways, alone and in combination.

Some of these ways include the battery ID codes. For instance, the processor 2230 can be configured to determine that the third battery C 2243 is presently not received in the unit receptacle 2202 by inputting the battery 1 ID code 2261 but not the battery 3 ID 2263 code from the unit receptacle 2202. For another instance, the charger memory 2228 can be further configured to store the battery 2 ID code 2262 when the second battery B 2242 is received in the charger receptacle 2222, the status indication 2227 can include the battery 2 ID code 2262, and the processor 2230 can be configured to determine that the third battery C 2243 is presently not received in the charger receptacle 2222 by parsing, from the status indication 2227, the battery 2 ID code 2262 but not the battery 3 ID code 2263.

Some of these ways include a battery state table 2278, which can be maintained in the unit memory 2208 or elsewhere. In particular, the unit memory 2208 can be configured to store a battery state table 2278 with battery data about the first battery A 2241, the second battery B 2242, and the third battery C 2243. The processor 2230 can be further configured to update the battery state table 2278 with battery data derived from at least the unit 2200, and identify the third battery C 2243 as unplugged from the battery state table 2278. Optionally and in addition, the unit 2200 may include a clock that is configured to provide time inputs, in which the processor 2230 is further configured to update the battery state table 2278 also with time inputs provided by the clock and corresponding to the battery data. Examples are now presented.

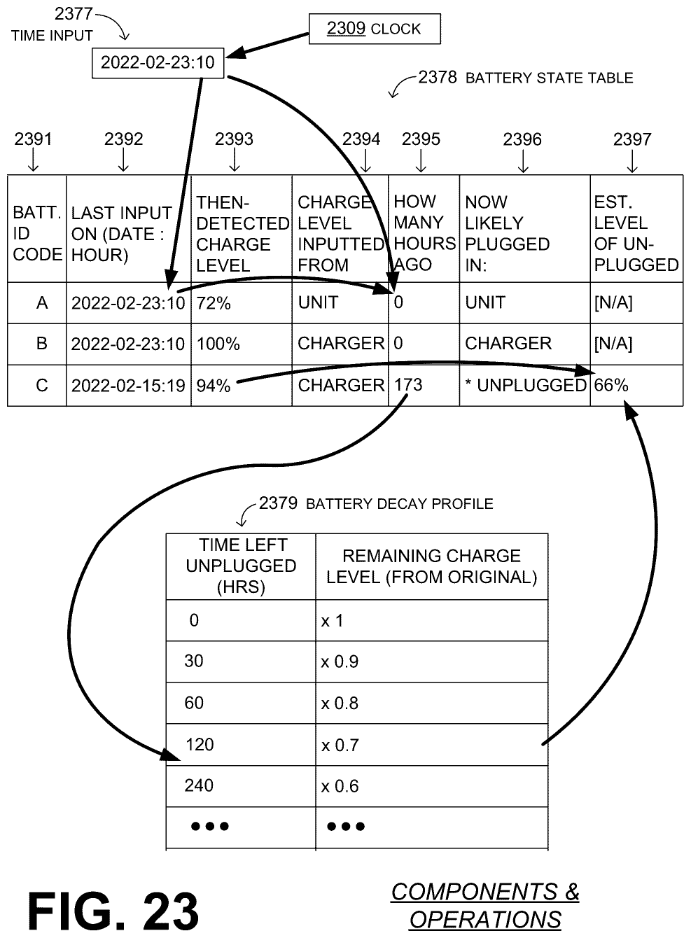

FIG. 23 is a diagram showing a sample clock 2309, a sample time input 2377, a sample battery state table 2378, and a sample battery decay profile 2379.

The clock 2309 can be configured to provide the time input 2377. In this example, the time input includes both a date (Feb. 23, 2022), and a time (10). In this example, the time shows the hours only, as the minutes and the seconds might not be necessary for this operation. In addition, the time is on a 24-hour basis, so “10” means 10 am, and “19” means 7 pm.

The battery state table 2378 has a headings row, and another three rows—one for the status of each of the three system batteries. In a column 2391, the battery ID codes are shown A, B and C. The next column 2392 shows the last time at which an input was received from that battery. This data has been filled or updated by the time input 2377, per an arrow landing on the row for battery A, column 2392. This data shows that the batteries A and B have been alternated, while the battery C has been plugged into neither receptacle since February 15, at 7 pm, which is more than a week prior. The next column 2393 shows the charge level detected at the time of the column 2392, as percentages. The next column 2394 shows which device the detected charge level was inputted from, at the time of the column 2392. The next column 2395 shows a detected elapsed time, as described later in this document. The next column 2396 shows the plug-in status, known for the batteries A and B, and inferred for the battery C. The battery C has been identified as unplugged. The last column 2397 shows the estimated charge level of the battery that is identified as unplugged.

Returning to FIG. 22, in embodiments the processor 2230 is further configured to determine how much time has elapsed since the third battery was last known to be received in the unit receptacle or in the charger receptacle. It may do so from the battery state table 2278. An example is seen in FIG. 23, with the detected elapsed time of the column 2395. This is expressed in terms of how many hours ago the detection was, from the time of the column 2392. The values of the column 2395 are produced from the present time input 2377, per an arrow landing on the row for battery A, and also from column 2392 by subtraction, per another arrow within the row for battery A. The answer is 0 hours for batteries A and B, and 173 hours for battery C.

Returning to FIG. 22, in embodiments the processor 2230 is further configured to determine, from the elapsed time, whether or not a reserve-battery alert criterion is met. This may be implemented in a number of ways. In some embodiments, the reserve-battery alert criterion includes that the elapsed time is longer than a threshold. For example, the threshold could be 120 hours, 350 hours, and so on. Such embodiments may have the drawback that they require reliance on assumptions which, without more, may not be true. For instance, an assumption can be that, when the third battery was last unplugged, it was fully charged. That may not be true. Or, the table 2230 may know that, and adjust accordingly. Another assumption may be that the storage temperature will be continuously temperate. If, however, the unplugged battery is stored in the trunk of a car that is routinely parked outdoors, prolonged spells of excessively high or low temperature may accelerate how fast the reserve battery's charge level decays.

In some embodiments, the processor 2230 is further configured to produce, at least from the elapsed time, an estimate of an amount of charge remaining stored in the third battery, such as the estimate 2233. In such embodiments, the reserve-battery alert criterion may include that the estimate is less than a low-reserve threshold, for example 66%, 50%, 40% or even less. In some of these embodiments, the estimate is produced also from a battery decay profile, such as the battery decay profile 2279. An example is now described.

Returning to FIG. 23, the sample battery decay profile 2379 is a table that has two columns. The left column relates to hours unplugged, and the right column relates to expected percent deterioration. In this example, the computed number of hours 173 in the column 2395 uses the profile 2379 to first look up the time left unplugged. Since 173 is more than 120 but less than 240, the row of 120 is selected, and the multiplier of the selected row (0.7) is thus looked up. This multiplier multiplies the 94% of the column 2393, to produce the estimate of 66% in the column 2397.

Returning to FIG. 22, in embodiments the processor 2230 is further configured to cause, responsive to the reserve-battery alert criterion being met, the UI 2280 to output a reminder prompt 2287 that has a content 2288 to charge also the unplugged third battery 2243 C. This, of course, above and beyond other prompts such as a low-charge prompt, which are not shown so as to not complicate this drawing further.

In some embodiments, the above-described no-swap criterion is also checked. In such embodiments, the status indication 2292 includes an indication of the second battery 2 level 2252, the processor 2230 is further configured to determine, from the indication of the second battery level, whether or not a no-swap criterion is met, and not cause, responsive to the no-swap criterion being met, the UI 2280 to output the reminder prompt 2287, even when the reserve-battery alert criterion is met. In such embodiments, an alternate or no criterion is caused to be output, and so on.

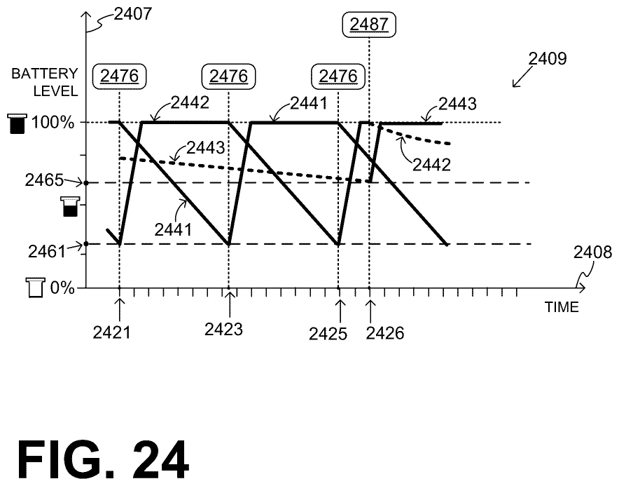

FIG. 24 is a timing diagram 2409 showing time evolutions of battery levels of the batteries in the embodiments of FIG. 22, for a specific scenario. The timing diagram 2409 uses a vertical axis 2407 for battery levels and a horizontal time axis 2408.

The time evolution of the battery level of the battery A 2241 is shown as a broken dark line 2441, the time evolution of the battery level of the battery B 2242 is shown as a broken dark line 2442, and the time evolution of the battery level of the battery C 2243 is shown as a broken dark line 2443.

All three of these lines 2441, 2442, 2443 are shown as a) solid when and where their values are directly known by the processor because their batteries are plugged into one of the receptacles, and as b) dotted when and where their values are not directly known by the processor, but they are subject to estimate.

At times 2421, 2423, 2425, the evolution of these diagrams is similar to those in FIG. 14. As such, low-charge prompts 2476 have been caused to be output at those times, because that is when the battery level of the battery in the unit 2200 just crosses below the first threshold 2461. During those times, the estimate for the third battery is shown as the dotted line 2443, slowly decreasing because unplugged.

In the scenario of FIG. 24, at a next time 2426 the reserve-battery alert criterion is met, for instance because the line 2443 may have reached down to the low-reserve threshold of the value 2465 (66%). If used, the no-swap criterion is not met at that time. At that time, a reminder prompt 2487 is caused to be output. The battery that is in the unit receptacle, which at the time is the second battery, is swapped for the third battery that was the reserve so far. The third battery is placed into the charger receptacle, while the charged second battery is maintained unplugged, as the new reserve battery. As such, past the time 2426, the line 2443 becomes solid because it is known, and increases relatively rapidly due to being charged by the charger. On the other hand, the line 2442 becomes dotted because it is no longer known. Nevertheless an estimate of it is shown, which decreases gradually due to being unplugged and not in service. In actual embodiments, the line 2443 may decay more gradually than is suggested in FIG. 24, but the example here was shown to illustrate the embodiments in operation.

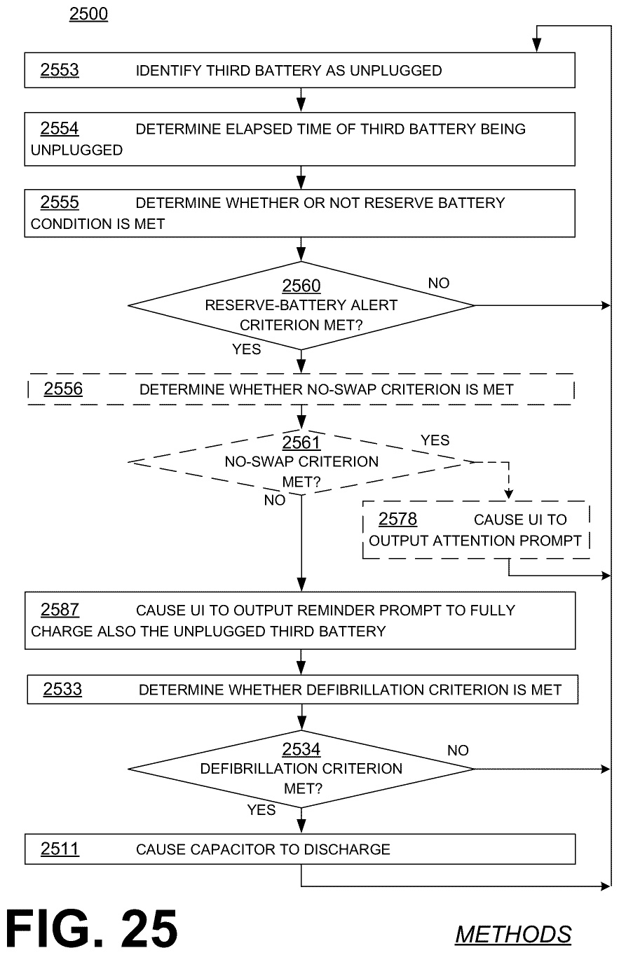

FIG. 25 shows a flowchart 2500 for describing methods according to embodiments. According to a first operation 2553, a third battery is identified as unplugged. This can be done in a number of ways, for instance responsive to determining that the third battery is presently not received either in a unit receptacle or in a charger receptacle.

According to another operation 2554, it may be determined how much time has elapsed since the third battery was last known to be received in the unit receptacle or in the charger receptacle.

According to another operation 2554, it may be determined whether or not a reserve-battery alert criterion is met.

According to another operation 2560, it may be inquired whether or not the reserve-battery alert criterion is met. The answer can be had from the operation 2555. If the answer is NO, then execution may return to the operation 2553.

If at the operation 2560 the answer is YES then, in some embodiments, the no-swap criterion is also implemented. For instance, according to another, optional operation 2556, it may be determined whether or not a no-swap criterion is met. The determination may be made from the second battery level, and possibly other data. Then according to another operation 2561, it may be inquired whether or not the no-swap criterion is met. The answer can be had from the operation 2556. If at the operation 2561 the answer is YES, then a UI can be caused to not output the reminder prompt. In fact, no prompt may be output. Or, according to another, optional operation 2578, an attention prompt may be caused to be output, before execution returns to the operation 2553.

If at the operation 2561 the answer is NO, or if the no-swap criterion is not implemented then, according to another operation 2587, a UI can be caused to output a reminder prompt to fully charge also a third battery that may have been unplugged for a long time.

Additional operations 2533, 2534 and 2511 may be performed as described respectively for the operations 1033, 1034 and 1011 of FIG. 10.

In some embodiments, a WCD includes circuitry to support regenerative charging of the WCD battery. Examples are now described.

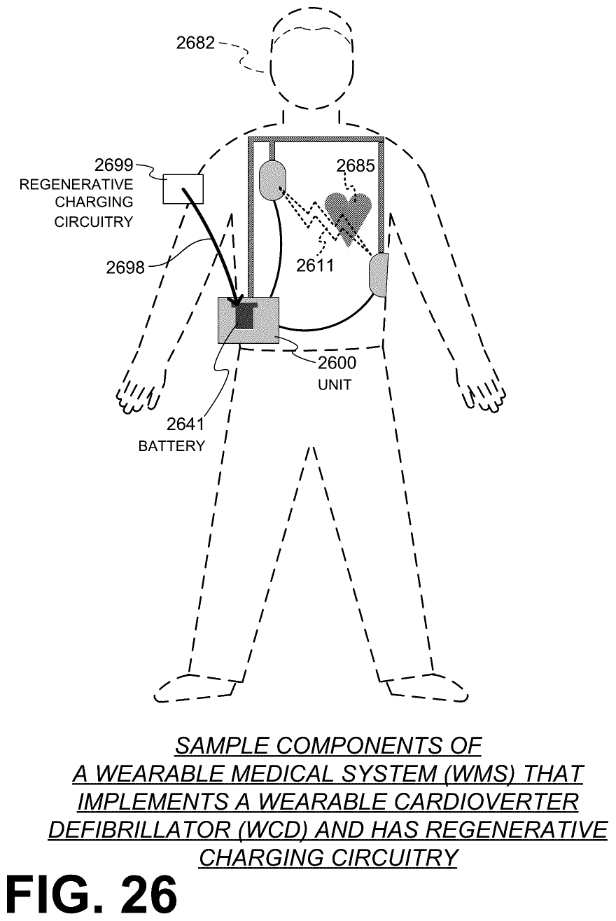

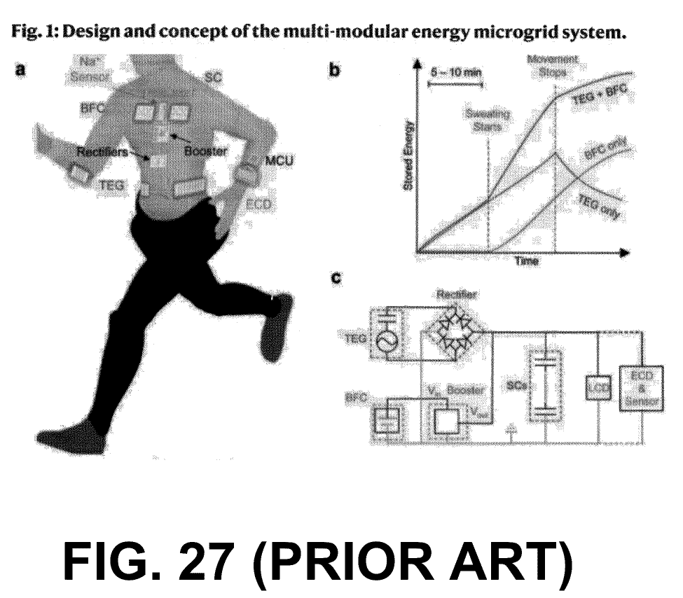

FIG. 26 shows a patient 2682 who is wearing a WMS that implements a WCD. The WMS of FIG. 26 can be in many ways similar to what is described in FIG. 1, and therefore many of its components are not described again. The WMS of FIG. 26 has a unit 2600, which is powered by a rechargeable battery 2641. In FIG. 26, the WMS is configured to administer a defibrillation shock 2611 though the heart 2685 of the patient 2682.

In addition, the WMS of FIG. 26 has regenerative charging circuitry 2699, which is configured to generate electrical charge and add it to the battery 2641 for recharging it. The recharging is shown by an arrow 2698 while, of course, electrical cabling will be used, and so on.

The regenerative charging circuitry 2699 can be implemented in a number of ways, for instance from converting movement by the patient to electrical energy (similar to a self-winding watch), converting heat generated by the patient's body to electrical energy, converting tension/compression of garment during use to electrical energy; converting sweat from the patient into electrical energy.

In an alternative approach, the WCD includes a charging port rather than removing the battery from the WCD to be charged in the charging station. Different power sources can be connected to the WCD's charging port. In embodiments that use the same battery as in the current WCD system, the charging source is a “smart” source that controls the charging process to avoid overheating, etc. The alternative power sources can be: a powered USB port from a car, airplane, portable power pack, etc.; a solar panel incorporated into/onto the carry pack; or from a smartphone, tablet, or laptop computer, for example.

Examples of wearable regenerative power technologies are described in “An electromagnetic rotational energy harvester using sprung eccentric rotor, driven by pseudo-walking motion”, accessible from: https://iss.mech.utah.edu/wp-content/uploads/sites/103/2018/12/1-52.0-S0306261918302186-main.pdf; “Stretchable helical architecture inorganic-organic hetero thermoelectric generator” accessible from: https://www.sciencedirect.com/science/article/abs/pii/S2211285516304712?via%3Dih ub#f0045.

A survey of possibilities can be found in: “11 of The Best Existing And in Development Wearable Technology That Create Power”, which is accessible from: https://interestingengineering.com/11-of-the-best-existing-and-in-development-wearable-technology-that-create-power. This survey recites that wearable technology is fast becoming ‘all the rage’ but current battery tech tends to limits its long-term usage. Wearable technology has been around for some time now but most electrical forms have one common unifying limitation, their batteries. These batteries tend to have limited capacities that directly impact their long-term use and once drained, the tech just becomes a fancy piece of jewelry. Several talented individuals around the world have been developing methods of harvesting power from an unlikely source—the wearer, or “you”. They range from tapping into your biochemistry to using your own motion to generate enough electricity to power electronic devices. If this technology can be mastered, we may see a time when batteries, at least for small personal devices, are a thing of the past.

1. Bionic Power Generates Power from Walking: Initially designed and built for use by the U.S. and Canadian Armies, Bionic Power has created a wearable technology, PowerWalk, for charging batteries. They believe that this technology will have great utility in disaster zones and remote working sites as well as recreational activities. PowerWalk is a light-weight piece of kit that generates electricity from the mechanical movement of the wearer whilst walking. This is much the same way as regenerative braking systems work. Generated power is then used to recharge the equipment's Li-ion (NiMH) batteries.

2. Piezoelectric Quartz Watches Are Still Some of the Best: Piezoelectric Quartz Watches, sometimes termed ‘automatic quartz’, have been around for some time now but remain one of the best, and stylish, wearables that generate power. They work by generating electricity using a piezoelectric quartz crystal and self-winding rotor. Electricity is generated from a rotating pendulum attached to a ‘large’ gear and small pinion setup that spins the pinion at high speed when the wearer moves. This motion is then translated to a miniature electrical generator that charges either a capacitor or rechargeable battery.

3. Solar and Motion Generating Cloth Could be the Future: Researchers at the Georgia Institute of Technology are developing a purely motion-generating electricity harvesting textile. Technically, it is bi-generational as it also integrates solar generation. Apart from direct solar generation, the textile generates electricity from the motion of the wearer from triboelectric nanogenerators. These generators work by combining the triboelectric effect and electrostatic induction to generate electricity from any movement that agitates the textile.

4. Could Mini-Solar Cell Textile Be the Future of Wearables: Researchers around the world, including in China, are developing polymer solar cell textiles that are able to generate small amounts of electricity. Like other entries on this list, successful creation of this kind of material could make batteries a thing of the past. Invented by Huisheng Peng, professor of macromolecular science at Fudan University in China, the polymer has an energy-conversion efficiency that varies less than 3% after bending for more than 200 cycles, researchers said.

5. Thermoelectrical Power Generation Makes Electricity From Your Own Body Heat: Researchers at the Korea Advanced Institute of Science and Technology (KAIST) have been working on a power generating wearable technology that harvests your body heat to make electricity. Using flexible thermoelectric (TE) power generation, it could be used to power future wearable devices.

6. iTeng Could Be the Future of Medical Devices: iTeng is another form of triboelectric nano-generation that harvests energy from the principle of static electricity. One interesting application of iTeng is that it produces electricity to power small medical devices like heart monitors. This kind of device has massive implications for future implantable devices and could, perhaps, be used in the future to self-power healthcare monitoring systems.

7. Yarn-like Rechargeable Zinc Batteries Are Almost Here: Researchers are currently developing a form of rechargeable zinc-ion battery that comes in an elastic yarn form. It is able to generate electricity whenever the yarn is bent, stretched, washed or cut. This material could, conceivably, form the basis for future ‘smart clothes’.

8. Electric Eel Inspired Fiber Capacitors: Chinese scientists have managed to produce a synthetic fiber that mimics bio-electrical abilities of electric eels. These stretchy eel-inspired fibers could be used to make self-powering wearable devices of the future. Being capacitors, they are able to store electrical charge on the surfaces of the conductors and can capture and release energy much more quickly than batteries can, although they usually store less energy than batteries do. Other researchers have found a way to print capacitors directly onto textiles too.

9. The Future of Shoes Could be Triboelectric: An obvious application of the principle of wearable power generation is to integrate the technology into the soles of your shoes. This is exactly what researchers at the Georgia Institute of Technology succeeded in creating. They were able to put piezoelectric technology into the heels of shoes that could harvest energy as the wearer walks or runs. Tests of the technology have shown that this a TENG-based shoe insole could produce a maximum output voltage and current density reached up to 220 V and 40 mA, respectively.

10. Your Sweatband Could Generate Electricity in the Future: An interesting potential future wearable technology could generate electricity as you sweat at the gym or on a hot day. Sweating releases lactate which can be used as ‘fuel’ molecules by enzymes to generate a small electrical current. These enzymes can be stuck onto nanotubes and formed into biofuel cells. These can then be woven into garments such as headbands and wristbands which can then readily generate enough electricity from chemical reactions with sweat lactate to power devices, like a watch. Other researchers have a similar concept using bacteria instead.

11. You Could Power Devices With Your Underwear or Trousers in the Future: Researchers have developed wearable piezoelectric energy harvesters that could be worn on your elbow or knee joints or trousers and underwear. Such wearable technology will enable the user to generate electricity whenever they move. Scientists note that we might not want to make these kinds of generators too efficient. Less is more, as the adage goes. MIT materials scientist and engineer Canan Dagdeviren explained that “it might do so by placing more of a load on the body, and you don't want it to make you tired”.

Another resource is the article: “‘Wearable Microgrid’ Uses the Human Body to Sustainably Power Small Gadgets”, accessible from: https://ucsdnews.ucsd.edu/pressrelease/wearable-microgrid-uses-the-human-body-to-sustainably-power-small-gadgets.

That article relates that nanoengineers at the University of California San Diego have developed a “wearable microgrid” that harvests and stores energy from the human body to power small electronics. It consists of three main parts: sweat-powered biofuel cells, motion-powered devices called triboelectric generators, and energy-storing supercapacitors. All parts are flexible, washable and can be screen printed onto clothing. The technology, reported in a paper published March 9 in Nature Communications, draws inspiration from community microgrids. “We're applying the concept of the microgrid to create wearable systems that are powered sustainably, reliably and independently,” said co-first author Lu Yin, a nanoengineering Ph.D. student at the UC San Diego Jacobs School of Engineering. “Just like a city microgrid integrates a variety of local, renewable power sources like wind and solar, a wearable microgrid integrates devices that locally harvest energy from different parts of the body, like sweat and movement, while containing energy storage.”

The wearable microgrid is built from a combination of flexible electronic parts that were developed by the Nanobioelectronics team of UC San Diego nanoengineering professor Joseph Wang, who is the director of the Center for Wearable Sensors at UC San Diego and corresponding author on the current study. Each part is screen printed onto a shirt and placed in a way that optimizes the amount of energy collected.

Biofuel cells that harvest energy from sweat are located inside the shirt at the chest. Devices that convert energy from movement into electricity, called triboelectric generators, are positioned outside the shirt on the forearms and sides of the torso near the waist. They harvest energy from the swinging movement of the arms against the torso while walking or running. Supercapacitors outside the shirt on the chest temporarily store energy from both devices and then discharge it to power small electronics.

Harvesting energy from both movement and sweat enables the wearable microgrid to power devices quickly and continuously. The triboelectric generators provide power right away as soon as the user starts moving, before breaking a sweat. Once the user starts sweating, the biofuel cells start providing power and continue to do so after the user stops moving.

“When you add these two together, they make up for each other's shortcomings,” Yin said. “They are complementary and synergistic to enable fast startup and continuous power.” The entire system boots two times faster than having just the biofuel cells alone, and lasts three times longer than the triboelectric generators alone.

The wearable microgrid was tested on a subject during 30-minute sessions that consisted of 10 minutes of either exercising on a cycling machine or running, followed by 20 minutes of resting. The system was able to power either an LCD wristwatch or a small electrochromic display—a device that changes color in response to an applied voltage—throughout each 30-minute session.

Greater Than the Sum of Its Parts: The biofuel cells are equipped with enzymes that trigger a swapping of electrons between lactate and oxygen molecules in human sweat to generate electricity. Wang's team first reported these sweat-harvesting wearables in a paper published in 2013. Working with colleagues at the UC San Diego Center for Wearable Sensors, they later updated the technology to be stretchable and powerful enough to run small electronics. The triboelectric generators are made of a negatively charged material, placed on the forearms, and a positively charged material, placed on the sides of the torso. As the arms swing against the torso while walking or running, the oppositely charged materials rub against each and generate electricity.

Each wearable provides a different type of power. The biofuel cells provide continuous low voltage, while the triboelectric generators provide pulses of high voltage. In order for the system to power devices, these different voltages need to be combined and regulated into one stable voltage. That's where the supercapacitors come in; they act as a reservoir that temporarily stores the energy from both power sources and can discharge it as needed.

Yin compared the setup to a water supply system: “Imagine the biofuel cells are like a slow flowing faucet and the triboelectric generators are like a hose that shoots out jets of water,” he said. “The supercapacitors are the tank that they both feed into, and you can draw from that tank however you need to.” All of the parts are connected with flexible silver interconnections that are also printed on the shirt and insulated by waterproof coating. The performance of each part is not affected by repeated bending, folding and crumpling, or washing in water—as long as no detergent is used.

The main innovation of that work is not the wearable devices themselves, Yin said, but the systematic and efficient integration of all the devices. “We're not just adding A and B together and calling it a system. We chose parts that all have compatible form factors (everything here is printable, flexible and stretchable); matching performance; and complementary functionality, meaning they are all useful for the same scenario (in this case, rigorous movement),” he said.

The paper: “A self-sustainable wearable multi-modular E-textile bioenergy microgrid system”, authored by Yin, L., Kim, K. N., Lv, J. et al., has its own FIG. 1 that shows a design and concept of a multi-modular energy microgrid system. That FIG. 1 is replicated in this document as FIG. 27, but with imperfections from converting the multi-colored drawing to the black and white required for this patent document.

FIG. 27 illustrates a wearable system that can harvest energy using thermoelectric triboelectric generators (TEGs) that harvest electricity on static electricity, and enzymatic or microbial fuel cells (BFCs) that can generate electricity from the patient's sweat. The TEG collected energy is rectified using bridge rectifies and stored in a supercapacitor (SC), while the BFC collected energy is boosted using a DC voltage booster and then stored in the SC. The other elements in this system are for sensors and displaying sensed data. Although not shown in the Figure above, the SC can be used to charge the WCDs battery, or alternatively, provide power in parallel with the WCD battery.

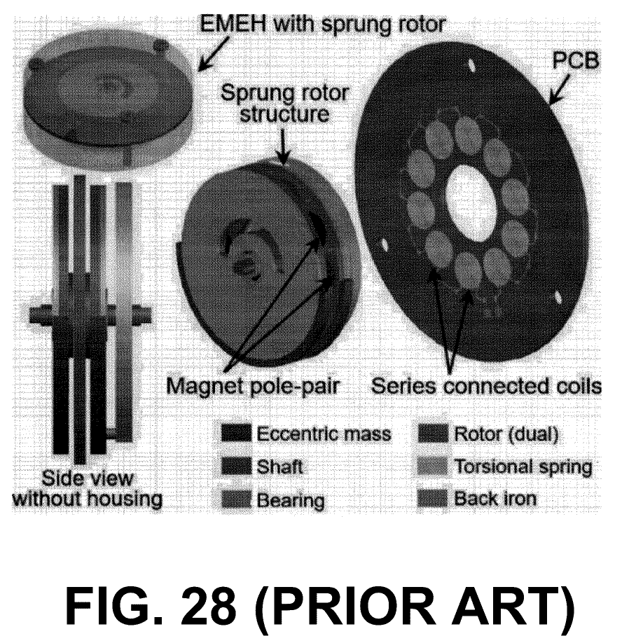

Further, the above system can be modified by adding a rotor assembly to convert patient motion (e.g., arm movement) into electrical energy. The rotor mechanism may be similar to that used in some self-winding watches but in this system the rotor generates electricity rather than winds a spring. An example is show in its own FIG. 2, taken from the article: M. A. Halim, R. Rantz, Q. Zhang, L. Gu, K. Yang, S. Roundy, An electromagnetic rotational energy harvester using sprung eccentric rotor, driven by pseudo-walking motion, Applied Energy, Volume 217, 2018, Pages 66-74, ISSN 0306-2619, https://doi.org/10.1016/j.apenergy.2018.02.093. That FIG. 2 is replicated in this document as FIG. 28, but with imperfections from converting the multi-colored drawing to the black and white required for this patent document.

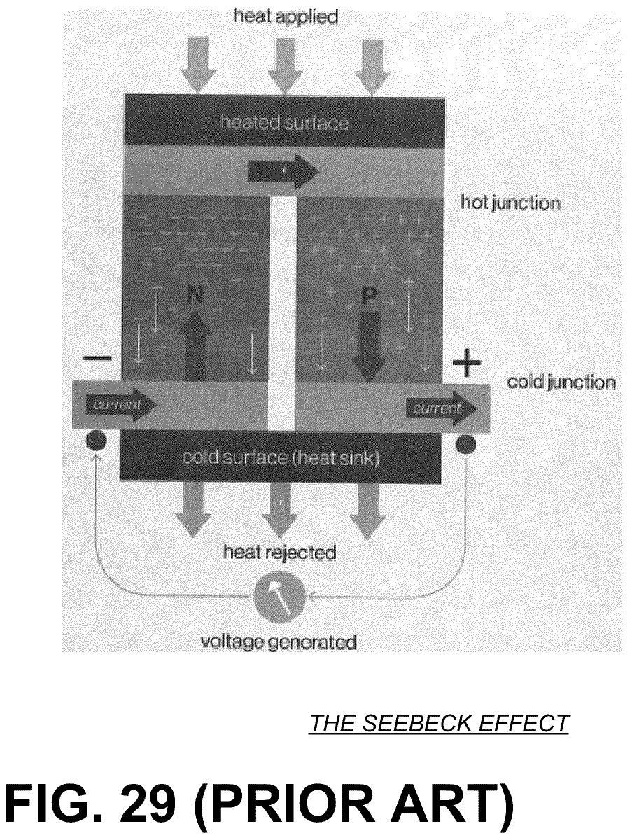

Still further, the above systems can be modified by adding a wearable thermoelectric generator. Some thermoelectric generators are based on the Seebeck effect. FIG. 29 illustrates the Seebeck effect in a diagram. The Seebeck effect is a phenomenon in which a temperature difference between two dissimilar electrical conductors or semiconductors produces a voltage difference between the two substances. When heat is applied to one of the two conductors or semiconductors, heated electrons flow toward the cooler conductor or semiconductor. If the pair is connected through an electrical circuit, direct current (DC) flows through that circuit. A heat differential between the “hot” side (which could be touching or facing the patient's body) and the cool side (which could be the side facing away from the patient and perhaps exposed to the external environment) can be used to generate a voltage at terminals. Note, if a voltage is applied to the terminals, it reverses the effect and generates a temperature difference that can be used to move heat from one side to the other to provide heating or cooling.

A flexible thermoelectric generator that can potentially be adapted for a wearable is disclosed in the article: Jhonathan P. Rojas, Devendra Singh, David Conchouso, Arpys Arevalo, Ian G. Foulds, Muhammad M. Hussain, Stretchable helical architecture inorganic-organic hetero thermoelectric generator, Nano Energy, Volume 30, 2016, Pages 691-699, ISSN 2211-2855, https://doi.org/10.1016/j.nanoen.2016.10.054.

Still further, the above systems can be modified by adding a piezoelectric devices to convert mechanical energy (e.g., motion from the patient's limbs while moving; compression from a patient putting his or her weight on the ground while walking or running; or stretching/tension/torsion of the WCD garment fabric) into electrical energy. U.S. Pat. No. 8,623,451 “LARGE-SCALE LATERAL NANOWIRE ARRAYS NANOGENERATORS” U.S. Pat. No. 10,536,098 “PIEZOELECTRIC ENERGY HARVESTER FOR HUMAN MOTION”, and U.S. Pat. No. 9,112,432 “PIEZOELECTRIC GENERATOR AND METHOD OF MANUFACTURING THE SAME” are just a few examples of patents directed to piezoelectric generators that can be adapted for a WCD garment.

Embodiments for Getting Energy from Sources other than the wearer: In these embodiments, the unit 2600 includes a charging port as necessary that can be connected to different power sources such as for example, a powered USB port from a car, airplane, portable power pack, etc.; a solar panel incorporated into/onto the carry pack; or a smartphone, tablet, or laptop computer. This approach can avoid the need for replaceable or swappable batteries that must be removed from the WCD to be charged in the charging station, as is required in currently available WCDs. Different power sources can be connected to the unit's charging port. In embodiments that use the same battery as in the current WCD system, the charging source is a “smart” source that controls the charging process to avoid overheating, etc. This is the system that is used in the current charging station—the battery is “dumb” and the charging station is “smart”. In alternative embodiments, the battery includes circuitry to sense temperature, voltage, etc. and to control the amount and/or rate of charge being loaded into the batter to ensure the battery doesn't overheat. This approach would allow the power source to be “dumb”. That is, for example, no modifications would be needed for a smartphone or laptop to charge the WCD battery.

In the methods described above, each operation can be performed as an affirmative act or operation of doing, or causing to happen, what is written that can take place. Such doing or causing to happen can be by the whole system or device, or just one or more components of it. It will be recognized that the methods and the operations may be implemented in a number of ways, including using systems, devices and implementations described above. In addition, the order of operations is not constrained to what is shown, and different orders may be possible according to different embodiments. Examples of such alternate orderings may include overlapping, interleaved, interrupted, reordered, incremental, preparatory, supplemental, simultaneous, reverse, or other variant orderings, unless context dictates otherwise. Moreover, in certain embodiments, new operations may be added, or individual operations may be modified or deleted. The added operations can be, for example, from what is mentioned while primarily describing a different system, apparatus, device or method.

A person skilled in the art will be able to practice the present invention in view of this description, which is to be taken as a whole. Details have been included to provide a thorough understanding. In other instances, well-known aspects have not been described, in order to not obscure unnecessarily this description.

Some technologies or techniques described in this document may be known. Even then, however, it does not necessarily follow that it is known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

1-183. (canceled)

184. A wearable medical system (“WMS”), the WMS comprising:
a sensor configured to sense a parameter of a patient;
a therapy electrode;
a first battery and a second battery, the first and the second batteries configured to store electrical charge, and to be rechargeable;
a charger having:
a charger receptacle configured to receive therein the first or the second battery, the charger configured to recharge the second battery when the second battery is received in the charger receptacle, and
a charger communication module configured to transmit a status indication about an operation of the charger;
a unit having:
a unit receptacle configured to receive therein the first or the second battery,
a unit communication module configured to receive the transmitted status indication,
a user interface (UI),
a capacitor electrically coupled to the therapy electrode, and
a processor configured to:
detect whether the unit communication module has received, within a time period, the transmitted status indication,
determine, from so detecting, whether or not a charger-not-charging condition is met,
cause, responsive to a determination that the charger-not-charging condition was met, the UI to output a prompt regarding the second battery not being charged by the charger,
determine, from the sensed parameter, whether or not a defibrillation criterion is met, and
cause, responsive at least in part to a determination that the defibrillation criterion was met, the capacitor to receive charge from the one of the first and the second battery that is received in the unit receptacle and then to discharge the received charge via the therapy electrode through a body of the patient; and
a support structure, coupleable to the unit, configured to be worn by the patient.

185. The WMS of claim 184, in which:
the unit communication module is configured to receive the transmitted status indication directly as transmitted from the charger communication module.

186. The WMS of claim 184, further including:
an electronic device distinct from the unit and from the charger, and
in which: the electronic device is configured to receive the status indication that is transmitted by the charger communication module,
the electronic device is further configured to retransmit the status indication it thus receives, and
the unit communication module is configured to receive the status indication that is retransmitted by the electronic device.

187. The WMS of claim 184, in which:
the charger-not-charging condition is met responsive to detecting that the unit communication module has not received the transmitted status indication within the time period.

188. The WMS of claim 184, in which:
the charger further has a charger memory configured to store a second battery level of an amount of charge stored in the second battery when the second battery is received in the charger receptacle,
the status indication includes an indication of the second battery level stored in the charger memory,
the charger communication module is further configured to transmit an updated status indication that includes an updated indication of the second battery level,
the charger-not-charging condition is met responsive to detecting that the updated indication of the second battery level is not larger than the indication of the second battery level.

189. The WMS of claim 184, in which:
the processor is further configured to:
determine whether or not a validation criterion is met, and
in which the caution prompt is caused to be output only when the validation criterion is met.

190. The WMS of claim 189, in which:
the processor is configured to determine whether or not the validation criterion is met responsive to detecting that a power-up routine has just been executed.

191. The WMS of claim 189, in which:
the processor is configured to determine whether or not the validation criterion is met responsive to detecting that a different battery is received in the unit receptacle than immediately before the power-up routine.

192. The WMS of claim 191, in which:
the unit also has a unit memory, and
the processor is configured to detect that a different battery is received in the unit receptacle by:
inputting a battery status datapoint of at least one of: a first level battery of an amount of charge stored in the first battery when the first battery is received in the unit receptacle, a first battery id code of the first battery when the first battery is received in the unit receptacle, and a time input of a time of the inputting or the storing of the first battery level or the first battery id code,
storing the inputted battery status datapoint in the unit memory,
again inputting the datapoint after the power-up event, and
comparing the again inputted battery status datapoint to the stored battery status datapoint.

193. A method for a wearable medical system (“WMS”) for a patient, the WMS including at least: a sensor configured to sense a parameter of the patient, a therapy electrode, a first battery and a second battery each configured to store electrical charge and to be rechargeable, a charger having: a charger receptacle configured to receive therein the first or the second battery, the charger configured to recharge the second battery when the second battery is received in the charger receptacle, and a charger communication module configured to transmit a status indication about an operation of the charger, a unit having: a unit receptacle configured to receive therein the first or the second battery, a unit communication module configured to receive the transmitted status indication, a user interface (UI), a capacitor electrically coupled to the therapy electrode, and a processor, the WMS further including a support structure, coupleable to the unit, configured to be worn by the patient, the method including at least:
detecting whether the unit communication module has received, within a time period, the transmitted status indication;
determining, from so detecting, whether or not a charger-not-charging condition is met;
causing, responsive to a determination that the charger-not-charging condition was met, the UI to output a prompt regarding the second battery not being charged by the charger;
determining, from the sensed parameter, whether or not a defibrillation criterion is met; and
causing, responsive at least in part to a determination that the defibrillation criterion was met, the capacitor to receive charge from the one of the first and the second battery that is received in the unit receptacle and then to discharge the received charge via the therapy electrode through a body of the patient.

194. The method of claim 193, in which:
the unit communication module is configured to receive the transmitted status indication directly as transmitted from the charger communication module.

195. The method of claim 193, in which:
the WMS further includes: an electronic device distinct from the unit and from the charger, the electronic device being configured to receive the status indication that is transmitted by the charger communication module, the electronic device being further configured to retransmit the received status indication, and
the unit communication module is configured to receive the retransmitted status indication.

196. The method of claim 193, further including:
the charger-not-charging condition is met responsive to detecting that the unit communication module has not received the transmitted status indication within the time period.

197. The method of claim 193, in which:
the charger further has a charger memory configured to store a second battery level of an amount of charge stored in the second battery when the second battery is received in the charger receptacle,
the status indication includes an indication of the second battery level stored in the charger memory,
the charger communication module is further configured to transmit an updated status indication that includes an updated indication of the second battery level,
the charger-not-charging condition is met responsive to detecting that the updated indication of the second battery level is not larger than the indication of the second battery level.

198. The method of claim 193, further including:
determining whether or not a validation criterion is met, and
in which the caution prompt is caused to be output only when the validation criterion is met.

199. A wearable medical system (“WMS”), the WMS comprising:
a sensor configured to sense a parameter of the patient;
a therapy electrode;
a first battery and a second battery, each configured to store electrical charge and to be rechargeable;
a charger having:
a charger receptacle configured to receive therein the first or the second battery, the charger configured to recharge the second battery when the second battery is received in the charger receptacle,
a charger memory configured to store a second battery level of an amount of charge stored in the second battery when the second battery is received in the charger receptacle,
a charger communication module configured to transmit a status indication that includes an indication of the second battery level;
a unit having:
a unit receptacle configured to receive therein the first or the second battery,
a unit communication module configured to receive the transmitted status indication,
a user interface (UI),
a capacitor electrically coupled to the therapy electrode,
a processor configured to:
input a first battery level of an amount of charge stored in the first battery when the first battery is received in the unit receptacle,
determine, from the inputted first battery level, whether or not a low-charge condition is met,
input the indication of the second battery level that is included in the received status indication,
determine, from the indication of the second battery level, whether or not a no-swap criterion is met, and
cause, responsive to a determination that the low-charge condition was met and responsive to a determination that the no-swap criterion was not met, the UI to output a prompt to swap the first battery with the second battery in the unit receptacle, but
not cause, responsive to a determination that the low-charge condition was met and responsive to a determination that the no-swap criterion being met, the UI to output the low-charge prompt,
determine, from the sensed parameter, whether or not a defibrillation criterion is met, and
cause, responsive to a determination that the defibrillation criterion was met, the capacitor to receive charge from the one of the first and the second battery that is received in the unit receptacle and then to discharge the received charge via the therapy electrode through a body of the patient; and
a support structure, coupleable to the unit, configured to be worn by the patient.

200. The WMS of claim 199, in which:
the unit communication module is configured to receive the transmitted status indication directly as transmitted from the charger communication module.

201. The WMS of claim 199, further including:
an electronic device distinct from the unit and from the charger, and
in which: the electronic device is configured to receive the status indication that is transmitted by the charger communication module,
the electronic device is further configured to retransmit the received status indication, and
the unit communication module is configured to receive the retransmitted status indication.

202. The WMS of claim 199, in which:
the low-charge condition includes that the inputted first battery level is below a first threshold.

203. The WMS of claim 199, in which:
the unit further has a unit memory configured to store the inputted first battery level and the inputted indication of the second battery level.

204. The WMS of claim 199, in which:
the no-swap criterion includes that the indication of the second battery level is lower than a threshold.

205. The WMS of claim 199, in which:
the no-swap criterion includes that the indication of the second battery level is lower than the inputted first battery level.
